(19) United States
(12) Patent Application Publication
(10) Pub. No.: US 2026/0308522 A1
(43) Pub. Date: Oct. 8, 2026

(54) CD4+ CYTOTOXIC T CELLS AND METHODS OF USE THEREOF

(71) Applicant: Dana-Farber Cancer Institute, Inc., Boston, MA (US)

(72) Inventor: Baochun Zhang, Newton, MA (US)

(21) Appl. No.: 19/483,069

(22) PCT Filed: May 13, 2024
(86) PCT No.: PCT/US2024/029045

§ 371 Date: Nov. 10, 2025

Related U.S. Application Data
(60) Provisional application No. 63/466,070, filed on May 12, 2023.
(60) Provisional application No. 63/637,675, filed on Apr. 23, 2024.

(51) Int. Cl. C12N 5/0783; A61K 9/00; A61K 35/17; A61K 39/00; A61K 40/11; A61K 40/42; A61P 35/00; C07K 14/705; C12N 5/00

(52) U.S. Cl. CPC C12N 5/0638; A61K 9/0019; A61K 35/17; A61K 39/00; A61K 40/11; A61K 40/42; A61P 35/00; C07K 14/70539; C12N 5/0087; A61K 2039/5158; C12N 2501/24; C12N 2501/599; C12N 2502/30; C12N 2506/11

(57) ABSTRACT

Provided herein are CD4+ cytotoxic T cells and methods of producing and using same.

### CROSS REFERENCE TO RELATED APPLICATIONS

This application claims priority to U.S. Provisional Application No. 63/466,070, filed May 12, 2023, and U.S. Provisional Application No. 63/637,675, filed Apr. 23, 2024, the disclosures of each of which are hereby incorporated by reference in their entirety.

### BACKGROUND

Despite extensive research and development of cancer therapies, including immune therapies such as PD-1 checkpoint blockade and CD19-directed chimeric antigen receptor (CAR)-T cell therapies, durable remission has been difficult to achieve in many patients. Tumor immunity has traditionally focused on the role of cytotoxic CD8+ T cells to identify and kill cancer cells. However, recent clinical studies have pointed out the presence and potential role of CD4+ cytotoxic T cells (CD4+ CTLs) in direct cancer-killing ability. However, methods for efficiently producing CD4+ CTLs, particularly those that are multi-antigen-targeted and useful for adoptive cell therapies, have not been available to date. Thus, there remains an ongoing need to develop methods for producing CD4+ CTLs and using the CD4+ CTLs, particularly in killing cancer cells and in treating cancer.

### SUMMARY

The present disclosure provides inter alia CD4+ cytotoxic lymphocytes (CTLs) and methods, uses, and technologies related thereto. In some embodiments, the present disclosure provides methods for producing CD4+ CTLs using antigen presenting cells (APCs), such as APCs derived from cancer cells, to activate CD4+ T cells. The methods comprise contacting a CD4+ cell or a population of CD4+ T cells with one or more target cells described herein. In some embodiments, the methods comprise contacting a CD4+ T cell or a population of CD4+ T cells with antigen presenting cell(s) (e.g., a cancer cell-derived APC, such as a major histocompatibility complex II (MHC II)-expressing cancer cell(s)) and one or more agents that promote co-stimulation of the CD4+ T cells. In some embodiments, co-stimulation is through CD27-mediated signaling (e.g., CD70/CD27 signaling). In some embodiments, co-stimulation is through OX40-mediated signaling (e.g., OX40L/OX40 signaling). In some embodiments, co-stimulation is through both CD27-mediated signaling (e.g., CD70/CD27 signaling) and OX40-mediated signaling (e.g., OX40L/OX40 signaling). Such methods can be used to generate CD4+ CTLs that target cancers cells, including cancer cells that do not express CD70 and/or OX40L.

In some embodiments, there are provided methods of producing CD4+ CTLs that comprise contacting CD4+ T cells with major histocompatibility complex II (MHC II)-expressing cancer cells and (i) one or more agents that promote CD27-mediated co-stimulation of the CD4+ T cells and/or (ii) one or more agents that promote OX40-mediated co-stimulation of the CD4+ T cells, under conditions and for a time suitable for the formation of at least one immune complex between the CD4+ T cells and the cancer cells and promotion of the CD27-mediated co-stimulation of the CD4+ T cells and/or OX40-mediated co-stimulation of the CD4+ T cells, thereby producing the CD4+ CTLs.

Any embodiment or number of embodiments described herein are combinable in any way.

For example, in some embodiments, there are provided methods that do not comprise contacting CD4+ T cells with an OX40-mediated co-stimulation agent. In some embodiments, provided are methods that do not comprise contacting CD4+ T cells with a CD27-mediated co-stimulation agent. In some embodiments, provided are methods that comprise contacting CD4+ T cells with one or more agents that promote CD27-mediated co-stimulation of the CD4+ T cells and one or more agents that promote OX40-mediated co-stimulation of the CD4+ T cells.

In some embodiments, one or more agents that promote CD27-mediated co-stimulation comprise the MHC II-expressing cancer cells, wherein the MHC II-expressing cancer cells are or have been engineered to express CD70 on their cell surface. In some embodiments, provided methods comprise contacting a CD4+ T cell or a population of CD4+ T cells with MHC II-expressing cancer cells that also express CD70. In some embodiments, MHC II-expressing cancer cells have been engineered to exogenously express CD70. In some embodiments, MHC II-expressing cancer cells express both CD70 and OX40L.

In some embodiments, one or more agents that promote CD27-mediated co-stimulation comprise a soluble CD70. In some embodiments, soluble CD70 comprises a CD70 fusion protein (e.g., a CD70-Fc fusion protein).

In some embodiments, one or more agents that promote CD27-mediated co-stimulation comprise an anti-CD27 agonistic antibody.

In some embodiments, one or more agents that promote OX40-mediated co-stimulation comprise the MHC II-expressing cancer cells, wherein the MHC II-expressing cancer cells are or have been engineered to express OX40 ligand on their cell surface. In some embodiments, provided methods comprise contacting a CD4+ T cell or a population of CD4+ T cells with MHC II-expressing cancer cells that also express OX40L. In some embodiments, MHC II-expressing cancer cells have been engineered to exogenously express OX40L. In some embodiments, MHC II-expressing cancer cells express both CD70 and OX40L.

In some embodiments, one or more agents that promote OX40-mediated co-stimulation comprise a soluble OX40 ligand. In some embodiments, a soluble OX40 ligand comprises an OX-40L fusion protein (e.g., an OX40L-Fc fusion protein).

In some embodiments, one or more agents that promote OX40-mediated co-stimulation comprise an anti-OX40 agonistic antibody.

In some embodiments, one or more agents for co-stimulating CD4+ T cells comprise a) the MHC II-expressing cancer cells, wherein the MHC II-expressing cancer cells are or have been engineered to express CD70 on their cell surface; b) a soluble CD70, optionally wherein the soluble CD70 comprises a CD70 fusion protein (e.g., a CD70-Fc fusion protein); c) an anti-CD27 agonistic antibody; d) the MHC II-expressing cancer cells, wherein the MHC II-expressing cancer cells are or have been engineered to express OX40 ligand (OX40L) on their cell surface; e) soluble OX40L, optionally wherein the soluble OX40L is an OX40L fusion protein (e.g., an OX40L-Fc fusion protein); and/or f) an anti-OX40 agonistic antibody.

In some embodiments, provided methods comprise culturing the CD4+ T cells with an anti-CD27 agonistic antibody. In some embodiments, the CD4+ cells are cultured with an anti-CD27 agonistic antibody for 1 day, 2 days, 3 days, 4 days, 5 days, 6 days, 7 days, 8 days, 9 days, 10 days, or more. In some embodiments, the CD4+ T cells are cultured in the presence of anti-CD27 agonistic antibody from the start of culturing (day 0). In some embodiments, the CD4+ T cells are cultured in the presence of anti-CD27 agonistic antibody for the entire duration of culturing. In some embodiments, anti-CD27 agonistic antibody is present for a portion of the culturing. In some embodiments, anti-CD27 agonistic antibody is added at a time after the start of culturing. In some embodiments, the CD4+ T cells are cultured in the presence of anti-CD27 agonistic antibody starting from day 1, 2, 3, 4, 5, or 6 of culturing.

In some embodiments, provided methods comprise culturing the CD4+ T cells with an anti-OX40 agonistic antibody. In some embodiments, the CD4+ cells are cultured with an anti-OX40 agonistic antibody for 1 day, 2 days, 3 days, 4 days, 5 days, 6 days, 7 days, 8 days, 9 days, 10 days, or more. In some embodiments, the CD4+ T cells are cultured in the presence of anti-OX40 agonistic antibody from the start of culturing (day 0). In some embodiments, the CD4+ T cells are cultured in the presence of anti-OX40 agonistic antibody for the entire duration of culturing. In some embodiments, anti-OX40 agonistic antibody is present for a portion of the culturing. In some embodiments, anti-OX40 agonistic antibody is added at a time after the start of culturing. In some embodiments, the CD4+ T cells are cultured in the presence of anti-OX40 agonistic antibody starting from day 1, 2, 3, 4, 5, or 6 of culturing.

In some embodiments, CD4+ T cells for use in producing CD4+ CTLs by methods described herein are CD4+ T cells, naïve CD4+ T cells, memory CD4+ T cells, central memory CD4+ T cells, effector memory CD4+ T cells, Trp1-specific CD4+ T cells, tumor antigen-specific CD4+ T cells, neoantigen-specific CD4+ T cells, CD4+ T cells derived from peripheral blood mononuclear cells (PBMCs), CD4+ T cells derived from cord blood, CD4+ T cells derived from tumor-infiltrating lymphocytes, primary CD4+ T cells, CD4+ T cells derived from a cell line, CD4+ cells expanded in vitro, and/or CD4+ cells activated in vitro.

In some embodiments, CD4+ T cells are allogeneic, syngeneic, or autologous to the APCs. In some embodiments, CD4+ T cells are allogeneic, syngeneic, or autologous to the MHC II-expressing cancer cells.

In some embodiments, CD4+ T cells are selected from the group consisting of mammalian CD4+ T cells, rodent CD4+ T cells, mouse CD4+ T cells, primate CD4+ T cells, and human CD4+ T cells.

In some embodiments, cancer cells for use in producing CD4+ CTLs by methods described herein are allogeneic, syngeneic, or autologous to the CD4+ T cells.

In some embodiments, APCs, such as cancer cells, are or have been induced to express MHC II through contact with interferon gamma (IFNγ). In some embodiments, cells are cultured in the presence of IFNγ at a dose of 10 ng/ml to 400 ng/ml. In some embodiments, cells are cultured in the presence of IFNγ at a dose of 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 150 ng/ml, 200 ng/ml, 250 ng/ml, 300 ng/ml, 350 ng/ml, or 400 ng/ml. In some embodiments, the cells are cultured with IFNγ for 1 day, 2 days, 3 days, 4 days, 5 days, or more. In some embodiments, IFNγ is human IFNγ. In some embodiments, cells are cultured in the presence of human IFNγ at a dose of 10 ng/ml to 200 ng/ml. In some embodiments, the cells are cultured with human IFNγ for 2 days to 4 days. In some embodiments, cells are cultured in the presence of human IFNγ at a dose of 20 ng/ml for 3 days. In some embodiments, IFNγ is mouse IFNγ. In some embodiments, cells are cultured in the presence of mouse IFNγ at a dose of 10 ng/ml to 400 ng/ml. In some embodiments, the cells are cultured with mouse IFNγ for 2 to 5 days. In some embodiments, cells are cultured in the presence of mouse IFNγ at a dose of 100 ng/ml for 4 days.

In some embodiments, cancer cells are or have been induced to express MHC II by expression of class II transactivator (CIITA).

In some embodiments, cancer cells endogenously (e.g., natively) express MHC-II. In some embodiments, cancer cells are derived from a cancer that endogenously (e.g., natively) expresses MHC II.

In some embodiments, provided methods comprise contacting the CD4+ cells with IL-2. Without being bound by theory, it is believed that IL-2 treatment may be useful in certain embodiments (e.g., when certain T cells have relatively low affinity T cell antigen binding) to promote CD4+ T cell survival and/or proliferation such that EOMES-mediated CD4+ cytotoxic T cell differentiation can be executed.

In some embodiments, cancer cells do not endogenously (e.g., do not natively) express CD70. In some embodiments, cancer cells are derived from a cancer that does not endogenously (e.g., do not natively) express CD70.

In some embodiments, cancer cells do not endogenously (e.g., do not natively) express OX40L. In some embodiments, cancer cells are derived from a cancer that do not endogenously (e.g., do not natively) express OX40L.

In some embodiments, cancer cells endogenously (e.g., natively) express CD70 and/or OX40L. In some embodiments, cancer cells are derived from a cancer that (e.g., natively) endogenously expresses CD70 and/or OX40L.

In some embodiments, cancer cells are derived from a lymphoma and/or a leukemia. In some embodiments, cancer cells are from a B cell leukemia, a B cell lymphoma, diffuse large B cell lymphoma (DLBCL), classical Hodgkin lymphoma (cHL), and/or an acute myeloid leukemia (AML). In some embodiments, cancer cells are derived from a solid tumor. In some embodiments, cancer cells are derived from a melanoma, bladder cancer, breast cancer, head and neck cancer, hepatocellular carcinoma (HCC), colon cancer,

In some embodiments, cancer cells present at least one MHC II-antigen complex on their cell surface. In some embodiments, an antigen presented by an MHC-II-antigen complex is a tumor-associated antigen and/or a neoantigen.

In some embodiments, CD4+ CTLs produced by methods provided herein express a T cell receptor (TCR) that specifically binds to an MHC II-antigen complex expressed by cancer cells on the cancer cell surface.

In some embodiments, CD4+ CTLs produced by methods provided herein express a granzyme, such as, e.g., granzyme B. In some embodiments, CD4+ CTLs produced by methods provided herein express a perforin. In some embodiments, CD4+ CTLs produced by methods provided herein express a granzyme and a perforin. In some embodiments, CD4+ CTLs produced by methods provided herein express CD69. In some embodiments, CD4+ CTLs produced by methods provided herein express a granzyme (e.g., granzyme B), a perforin, and CD69. In some embodiments, CD4+ CTLs produced by methods provided herein are Eomes-programmed.

In some embodiments, CD4+ CTLs provided herein secrete cytokine. In some embodiments, CD4+ CTLs secrete IFNγ.

In some embodiments, CD4+ CTLs provided herein induce an antigen-specific T cell immune response. In some embodiments, CD4+ CTLs provided herein induce a multi-antigen specific T cell immune response.

In some embodiments, CD4+ CTLs provided herein are capable of killing cancer cells.

In some embodiments, CD4+ CTLs provided herein directly kill cancer cells that express on their cell surface the MHC II-antigen complex specifically recognized by the CD4+ CTLs

In some embodiments, CD4+ CTLs provided herein proliferate. In some embodiments, CD4+ CTLs provided herein can be expanded in vitro and/or ex vivo.

In some embodiments, CD4+ CTLs provided herein persist in vivo. In some embodiments, CD4+ CTLs provided herein persist in vivo for at least 6 months, at least 7 months, at least 8 months, at least 9 months, at least 10 months, at least 11 months, at least 1 year, at least 1.5 years, at least 2 years, at least 3 years, at least 4 years, at least 5 years, at least 6 years, at least 7 years, at least 8 years, at least 9 years, or at least 10 years. In some embodiments, CD4+ CTLs provided herein persist in vivo for a duration that is between 6 months to 10 years, or any range in between inclusive, such as 6 months to 12 months, 6 months to 2 years, 6 months to 5 years, and the like.

In some embodiments, CD4+ CTLs provided herein execute CD4+ helper function. In some embodiments, CD4+ helper function comprises (i) licensing APC to primer anti-cancer CD8+ CTLs, (ii) activating anti-cancer NK cells, (iii) activating anti-cancer macrophages, (iv) activating anti-cancer CD8+ T cells, and/or (v) activating anti-cancer immune cells. Such CD4+ CTL-mediated helper function can thereby kill MHC-II negative cancer cells.

In some embodiments, provided methods further comprise culturing the CD4+ CTLs under conditions suitable to allow proliferation of the CD4+ CTLs. In some embodiments, CD4+ CTLs are cultured for a period of at least 1 day, at least 2 day, at least 3 days, at least 4 days, at least 5 days, at least 6 days or at least 7 days. In some embodiments, CD4+ CTLs are cultured for a period selected from the group consisting of 1 day, 2 days, 3 days, 4 days, 5 days, 6 days, 7 days, 8 days, 9 days, 10 days, 11 days, 12 days, 13 days, and 14 days.

In some embodiments, culturing the CD4+ CTLs under conditions suitable to allow proliferation of the CD4+ CTLs comprises culturing with IL-2. In some embodiments, the IL-2 is added to CD4+ CTL cultures after 36 hours, 48 hours, 60 hours, 72 hours, or 84 hours of culturing, In some embodiments, IL-2 is added to CD4+ CTL cultures after about 2 to 4 days of culturing.

In some embodiments, provided methods further comprise a step of treating the APCs (e.g., cancer cells) to prevent further proliferation before contact with the CD4+ T cells. In some embodiments, provided methods further comprise irradiating the APCs (e.g., cancer cells).

In some embodiments, provided methods further comprise a step of isolating and/or purifying the CD4+ CTLs.

In some embodiments, provided methods are performed ex vivo or in vitro. In some embodiments, CD4+ T cells are contacted ex vivo or in vitro.

In some embodiments, the present disclosure provides compositions comprising CD4+ CTLs produced according to methods described herein.

In some embodiments, provided compositions comprise CD4+ CTLs that are specific for antigens expressed by the cancer cells and presented by MHC II. In some embodiments, provided compositions comprise CD4+ CTLs that are multi-specific for antigens expressed by the cancer cells and presented by MHC II.

In some embodiments, provided are immunogenic compositions comprising CD4+ CTLs produced according to method described herein. In some embodiments, provided are vaccines comprising CD4+ CTLs produced according to method described herein. In some embodiments, provided immunogenic compositions and/or vaccines further comprise an adjuvant.

In some embodiments, an immunogenic composition or vaccine is capable of eliciting an immune response against antigen-expressing cancer cells in a subject. In some embodiments, an immune response is i) a T cell response, a CD4+ helper T cell response, a CD4_cytotoxic T cell response, and/or a CD8+ T cell response and/or ii) selected from the group consisting of T cell expansion, cytokine release, and/or cytotoxic killing.

In some embodiments, the present disclosure provides methods of killing cancer cells that comprise contacting cancer cells with a composition comprising CD4+ CTLs as described herein. In some embodiments, the present disclosure provides methods of killing cancer cells that comprise contacting cancer cells with a an immunogenic composition or vaccine comprising CD4+ CTLs as described herein.

In some embodiments, the present disclosure provides methods of treating cancer comprising administering to a subject a composition comprising CD4+ CTLs as described herein. In some embodiments, the present disclosure provides methods of treating cancer that comprise contacting cancer cells with a an immunogenic composition or vaccine comprising CD4+ CTLs as described herein.

In some embodiments, the CD4+ CTLs provided herein are suitable for transplantation into a subject. In some embodiments, the CD4+ CTLs are allogeneic, syngeneic, or autologous to the cancer cells. In some embodiments, the CD4+ CTLs are allogeneic, syngeneic, or autologous to a subject.

In some embodiments, cancer cells are derived from a lymphoma and/or a leukemia. In some embodiments, cancer cells are from a B cell leukemia, a B cell lymphoma, diffuse large B cell lymphoma (DLBCL), classical Hodgkin lymphoma (cHL), and/or an acute myeloid leukemia (AML). In some embodiments, cancer cells are derived from a solid tumor. In some embodiments, cancer cells are derived from a melanoma, bladder cancer, breast cancer, head and neck cancer, hepatocellular carcinoma (HCC), colon cancer,

In some embodiments, provided are methods for treating cancer in a subject, where the subject has been or will be treated with an immune checkpoint inhibitor and/or treated for depletion of CD4+ regulatory T cells. In some embodiments, CD4+ CTLs provided herein contact the cancer cells in vivo, ex vivo, or in vitro. In some embodiments, CD4+ CTLs are MHC matched to the subject having the cancer.

In some embodiments, provided methods of treating cancer and/or killing cancer cells include CD4+ CTLs contacting cancer cells in vivo. In some embodiments, CD4+ CTLs induce an antigen-specific T cell immune response. In some embodiments, CD4+ CTLs are administered in a therapeutically effective amount to treat the cancer.

In some embodiments, a subject is an animal model of a cancer. In some embodiments a subject is a mammal. In some embodiments, a mammal is a human, a primate, a rodent, or a mouse.

In some embodiments, provided methods of treating cancer and/or killing cancer cells include further administering one or more adjuvants.

In some embodiments, provided methods of treating cancer and/or killing cancer cells include further administration of one or more additional therapies, such that the subject receives treatment with both CD4+ CTLs and the one or more additional therapies.

In some embodiments, provided methods of treating cancer and/or killing cancer cells include further administration of one or more immune checkpoint inhibitor therapies. In some embodiments, an immune checkpoint therapy regulates PD-1, PD-L1, PD-L2, LAG3, TIM3, CEACAMI, IDO, and/or CTLA4.

In some embodiments, provided methods of treating cancer and/or killing cancer cells include further administration of one or more immune co-stimulatory therapies. In some embodiments an immune co-stimulatory therapy promotes co-stimulation of the CD27 and/or OX40 co-stimulatory pathway(s) in the CD4+ CTLs.

In some embodiments, one or more regulatory T cell (Treg) depleting or inhibiting therapies are further administered.

In some embodiments, one or more additional therapies are administered concurrently or sequentially with the CD4+ CTLs.

### BRIEF DESCRIPTION OF THE DRAWINGS

The Drawing included herein are for illustration purposes only and not for limitation.

FIG. 1 provides exemplary schematics of tumor cell mediated activation of CD4+ T cells to generate CD4+ CTLs. The left panel depicts activation by a B-cell malignancy that expresses MHC-II and a costimulatory signal (CD70 and/or OX40L). The right panel depicts activation by other cancer cells that have been induced to express MHC-II by IFN-γ and a costimulatory signal (CD70 and/or OX40L).

FIG. 2A depicts FACS analysis of MHC-II and CD70 in A20 cells with or without CD70 transfection.

FIG. 2B depicts FACS analysis of Eomes, Granzyme B and IFN-γ in CD4+ T cells primed by irradiated CD70-transfected A20 cells for 6 days.

FIG. 2C depicts FACS analysis of Eomes, Granzyme B and IFN-γ in CD4+ T cells primed by irradiated CD70-transfected A20 cells for 9 days.

FIG. 3 depicts killing activity of CD4+ T cells against WT A20 and MHCII-null A20 target cells.

FIG. 4 depicts an exemplary schematics of tumor cell (e.g., melanoma) mediated activation of CD4+ T cells to generate CD4+ CTLs. The top panel depicts activation by a melanoma with IFN-γ induced expression of MHC-II and a costimulatory signal (CD70 and/or OX40L). The bottom panel depicts activation by a melanoma with IFN-γ induced expression of MHC-II and treatment with anti-CD27 and/or anti-OX40 antibodies for a costimulatory signal.

FIG. 5A depicts FACS analysis of MHC-II and CD70 in B16 cells with or without IFN-γ treatment or CD70 transfection.

FIG. 5B depicts FACS analysis of activation marker CD69 in Trp1-specific CD4+ T cells stimulated by the indicated B16 variants for 18 hours.

FIG. 5C depicts FACS analysis of Eomes expression in Trp1-specific CD4+ T cells primed by MHC-II⁺CD70⁺ B16 cells for 6 days, compared with naïve CD4 cells from a normal mouse.

FIG. 6A-FIG. 6E depict FACS analysis of naïve polyclonal CD4 cells co-cultured with irradiated MHC-II⁺CD70⁺ or MHC-II⁺OX40L⁺ B16 cells. FIG. 6A depicts FACS analysis of Eomes expression in unprimed naïve polyclonal CD4 (left), naïve polyclonal CD4 cells primed by MHC-II⁺CD70⁺ B16 cells (center) and naïve polyclonal CD4 cells primed by MHC-II⁺OX40L⁺ B16 cells (right). FIG. 6B-FIG. 6E depict FACS analysis of expression of perforin (FIG. 6B), granzyme B (FIG. 6C), IFN-γ (FIG. 6D), and PD-1 (FIG. 6E). Unprimed CD4 cells were used as a negative control.

FIG. 7A-FIG. 7C depicts FACS analysis of naïve polyclonal CD4 cells co-cultured with MHC-II⁺ B16 cells and with αCD27 or αOX40 agonistic antibody. FIG. 7A depicts FACS analysis of Eomes expression in unprimed naïve polyclonal CD4 (left), naïve polyclonal CD4 cells primed by MHC-II⁺ B16 cells and αCD27 agonistic antibody (center) and naïve polyclonal CD4 cells primed by MHC-II⁺ B16 cells and αOX40 agonistic antibody (right). FIG. 7B and FIG. 7C depict FACS analysis of expression of perforin and granzyme B, respectively. Unprimed CD4 cells were used as a negative control.

FIG. 8 depicts FACS analysis of naïve polyclonal CD4 cells co-cultured with MHC-II⁺ B16 cells and αCD27 or αOX40 agonistic antibody starting at different time points. Panels going from left to right depict CD4 cells co-cultured with MHC-II⁺ B16 cells with αCD27 agonistic antibody starting on day 0 (left most panel), with αCD27 agonistic antibody starting on day 3 (second from left), with αOX40 agonistic antibody starting on day 0 (second from right), and with αOX40 agonistic antibody starting on day 3 (right most panel).

FIG. 9A depicts a schematic of an adoptive cell therapy (ACT) protocol used herein. B16 refers to B16 melanoma cells; TBI refers to total body irradiation.

FIG. 9B depicts mean tumor volumes in the indicated groups of mice, n=3 mice per group. Statistics by one-way ANOVA, *, p<0.05.

FIG. 9C shows representative images from day 18 of tumors treated with the indicated CD4 cells according to the ACT protocol of FIG. 9A. Left image shows a representative “no ACT” control mouse; center image shows a representative mouse treated with naïve CD4 cells; right image shows a representative mouse treated with CD4 CTLS. Tumor burden is circled in each image.

FIG. 10A depicts FACS analysis of HLA-II expression on patient-derived melanoma cells with or without IFN-γ treatment (hIFNg 20 ng/ml for 3 days).

FIG. 10B depicts FACS analysis of patient-derived melanoma cells with or without CD70 transduction.

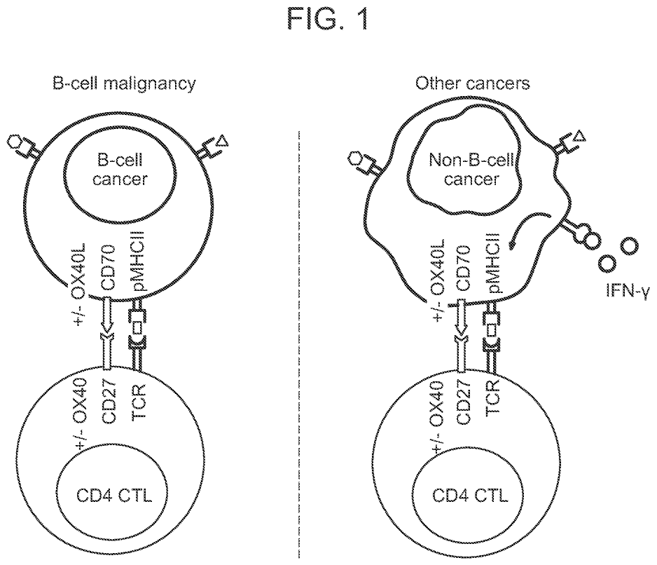

For any figure showing a bar histogram, curve, or other data associated with a legend, the bars, curve, or other data presented from left to right for each indication correspond directly and in order to the boxes from top to bottom, or from left to right, of the legend unless indicated otherwise.

### Certain Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

In this application, unless otherwise clear from context, (i) the terms “a” and “an” are used herein to refer to one or to more than one (i.e., to at least one) of the grammatical object of the article; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

The term “administering” means providing a pharmaceutical agent or composition to a subject, and includes, but is not limited to, administration by a medical professional and self-administration. This involves the physical introduction of a composition comprising a therapeutic agent to a subject, using any of the various methods and delivery systems known to those skilled in the art. In some embodiments, routes of administration for CD4+ CTLs described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, the term “antigen” is defined as a molecule that provokes an immune response. This immune response may involve either antibody production, or the activation of specific immunologically competent cells, or both.

As used herein, the term “autologous” is meant to refer to any material derived from the same subject to whom it is later to be re-introduced. A cell is considered “autologous” to a subject, if the cell is derived from the subject to whom it will be later re-introduced.

As used herein, the term “allogeneic” refers to any material derived from a genetically different subject of the same species. A cell is considered “allogeneic” with respect to a subject if the cell is derived from the same animal species as the subject but presents sequence variation in at least one genetic locus when compared to the subject's respective genetic locus.

The terms “cell,” “cell line,” and “cell culture” as used herein include progeny, which are any and all subsequent generations. It is understood that all progeny may not be identical due to deliberate or inadvertent mutations.

Generally, the term “cytotoxic T cell (CTL)” refers to T lymphocytes that can kill cells expressing an MHC-presented antigen, such as cells infected by viruses or transformed cancer cells. Herein, cytotoxic T cells include CD8+ T cells (CD8+ CTLs) and a subtype of CD4+ T cells (CD4+ CTLs). CTLs have specificity for peptide antigens that are presented in association with proteins encoded by the MHC genes and which are expressed on the surfaces of cells. In some embodiments, CTLs lyse cancer cells.

The term “exogenous” as used herein with reference to a nucleic acid and a particular cell refers to any nucleic acid that does not originate from that particular cell as found in nature. Thus, a non-naturally-occurring nucleic acid is considered to be exogenous to a cell once introduced into the cell. Nucleic acids that are naturally occurring also can be exogenous to a particular cell. For example, a unique nucleic acid sequence isolated from a cell of subject X is exogenous with respect to a cell of subject Y once that nucleic acid is introduced into a cell of subject Y.

The term “immune checkpoints” refers to a class of molecules on the surface of CD4+ and/or CD8+ T cells that fine-tune immune responses by down-modulating or inhibiting an anti-tumor immune response. Immune checkpoint proteins and their sequences are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragments, as well as nucleic acids encoding full-length immune checkpoint proteins and biologically active protein fragments thereof. In some embodiments, the term further encompasses any fragment according to homology descriptions provided herein. In one embodiment, the immune checkpoint protein is PD-1.

The term “immunotherapy” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agents for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy may be “untargeted,” which refers to administration of agents that do not selectively interact with immune system cells, yet modulates immune system function. Representative examples of untargeted therapies include, without limitation, chemotherapy, gene therapy, and radiation therapy.

The term “MHC match” or “HLA match” refers to a match between one or more of the MHC antigens (HLA antigens in humans) between a donor cell and a host recipient. Donor-recipient pairs of increasing numbers of MHC (HLA) matches (i.e. 1, 2, 3, 4, 5, and up to 6 allelic matches) are increasingly less likely to have host recipient immune cells, such as T cells and NK cells, recognize donor cells as foreign, and thus reduce the likelihood of an immune response against the donor.

The term “nucleic acid” or “polynucleotide” refers to deoxyribonucleic acids or ribonucleic acids and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al, Mol. Cell. Probes 8:91-98 (1994)).

The term “promoter” refers to a nucleic acid sequence, usually found upstream (5′) to a coding sequence, which directs transcription of a nucleic acid sequence into mRNA. The promoter or promoter region typically provide a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

A “subject” encompasses, but is not limited to, a mammal, e.g. a human, a domestic animal or a livestock including a cat, a dog, a cattle and a horse. As used herein the term “patient” without further designation is intended to encompass any warm blooded vertebrate domesticated animal (including for example, but not limited to livestock, horses, cats, dogs and other pets) and humans.

As used herein, the term “syngeneic” refers to any material derived from a genetically similar or identical subject. A cell is considered “syngeneic” with respect to a subject if the cell is sufficiently identical and immunologically compatible as to allow for transplantation.

The term “transfection” or “transduction” as used herein refers to a process by which exogenous nucleic acid is transferred or introduced into the host cell. A transfected or transduced cell is one which has been transfected or transduced with exogenous nucleic acid. The “transfected or transduced cell” includes the primary host cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, circular RNA, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Furthermore, in accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### DETAILED DESCRIPTION

CD4+ T cells have been best known for their helper functions, by which they can enlist and/or facilitate other immune effectors to fight cancers. Toes et al., Semin. Immunol. 10, 443-448 (1998). Specifically, CD4+ T cells can recognize tumor antigens presented by antigen-presenting cells (APCs) and help initiate an anti-tumor CD8+ T cell response by cross-priming or activate tumoricidal macrophages and NK cells by elaborating IFN-γ. Id. CD4+ T cells with cytotoxic function (CD4+ CTLs) are being increasingly recognized to arise in response to viral infections, as well as in various cancers, in both mice and humans. Swain et al., Nat. Rev. Immunol. 12, 136-148 (2012); Cenerenti et al., Front. Immunol. 13, 867189 (2022); Oh et al., Cell 181, 1612-1625 e1613 (2020); and Cachot et al., Sci Adv. 7 (2021).

For example, clinical responses to PD-1 blockade in patients with classic Hodgkin lymphoma (cHL) and melanoma are associated with expression of MHC-II on tumor cells, suggesting therapeutic importance of CD4+ T cells. Roemer et al. J. Clin. Oncol. 36, 942-950 (2018); Rodig et al. Sci. Transl. Med. 10 (450) (2018); Johnson et al. Nat Commun 7:10582 (2016).

A study of CD19 CAR-T cell therapy in long-surviving patients revealed two distinct phases of anti-tumor response: an initial phase dominated by CD8+ CAR-T cells, followed by the predominance (near 100%) of CD4+ CAR-T cells with cytotoxic characteristics in the ensuing years, suggesting that CD4+ T cells may be able to persist in vivo to provide long-lasting immunity. Melenhorst et al. Nature 602, 503-509 (2022). In a PTLD trial, clinical responses were found to significantly correlate with the frequency of CD4+ T cells in the infused T cells; CD4+ CTLs exhibited superior therapeutic efficacy over CD8+ CTLs in murine B-cell lymphoma models. Haque et al. Blood (2007); Choi et al. PNAS (2018). CD4+ CTLs can attack other cancers that upregulate MHC-II upon exposure to IFN-γ, including melanoma, bladder cancer, breast cancer, head and neck cancer, and hepatocellular carcinoma, as demonstrated in murine models and patients. Quezada et al. J. Exp. Med. 207, 637-650 (2010); Xie et al. J. Exp. Med. 207, 651-667 (2010); Kitano et al. Cancer Immunol. Res. 1, 235-244 (2013); Oh et al., Cell 181, 1612-1625 e1613 (2020); and Cachot et al., Sci Adv. 7 (2021). Together, these results suggest that CD4+ CTLs may be a useful cell-based therapy for treating cancers.

Recently, a method of eliciting a CD4+ CTL response using B cells that express the Epstein-Barr virus (EBV)-encoded signaling protein LMP1, which has a variety of effects, was described. Choi et al. PNAS 115, E686-E695 (2018) and Choi et al. Nature 590, 157-162 (2021). Though exceptionally effective for the instance wherein LMP1 is expressed, not all cancer cells do so. Identification of other CD4+ CTL production methods, such as signal 2 pathways that result in the production of CD4+ CTLs, were desired.

Moreover, developing CD4+ CTLs effective for solid tumors has been an ongoing challenge.

The present disclosure provides, among other things, CD4+ cytotoxic T lymphocytes (CTLs) and methods, uses, and technologies related thereto. The present disclosure demonstrates that provided CD4+ CTL are effective across tumor types, including solid tumors.

Methods of Producing CD4+ Cytotoxic Lymphocytes from CD4+ T Cells

In some embodiments, provided are methods of producing CD4+ CTLs that comprise contacting CD4+ T cells with major histocompatibility complex II (MHC II)-expressing cancer cells and (i) one or more agents that promote CD27-mediated co-stimulation and/or (ii) OX40-mediated co-stimulation of the CD4+ T cells under conditions and for a time suitable for the formation of at least one immune complex between the CD4+ T cells and the cancer cells and promotion of the CD27-mediated co-stimulation of the CD4+ T cells and/or OX40-mediated co-stimulation of the CD4+ T cells, thereby producing the CD4+ CTLs.

In some embodiments, provided are methods do not comprise contacting CD4+ T cells with an OX40-mediated co-stimulation agent. In some embodiments, provided are methods do not comprise contacting CD4+ T cells with a CD27-mediated co-stimulation agent. In some embodiments, provided are methods comprise contacting CD4+ T cells with one or more agents that promote CD27-mediated co-stimulation and one or more agents that promote OX40-mediated co-stimulation. In some embodiments, provided methods further comprise contacting with IL-2.

For example, provided are methods of producing CD4+ CTLs that comprise contacting CD4+ T cells with major histocompatibility complex II (MHC II)-expressing cancer cells and (i) one or more agents that promote CD27-mediated co-stimulation of the CD4+ T cells under conditions and for a time suitable for the formation of at least one immune complex between the CD4+ T cells and the cancer cells and promotion of the CD27-mediated co-stimulation of the CD4+ T cells, thereby producing the CD4+ CTLs. In some embodiments, provided methods further comprise contacting with IL-2.

In some embodiments, one or more agents that promote CD27-mediated co-stimulation comprise the MHC II-expressing cancer cells, wherein the MHC II-expressing cancer cells are or have been engineered to express CD70 on their cell surface. In some embodiments, the provided methods comprise contacting a CD4+ T cell or a population of CD4+ T cells with MHC II-expressing cancer cells that also express CD70. In some embodiments, MHC II-expressing APCs, such as cancer cells, are or have been engineered to exogenously express CD70. In some embodiments, MHC II-expressing cancer cells express both CD70 and OX40L.

In some embodiments, provided are methods of producing CD4+ CTLs that comprise contacting CD4+ T cells with one or more populations of cancer cells that present an antigen by a class II MHC molecule and express CD70 and/or OX40L, under conditions and for a time suitable for the formation of at least one immune complex between the CD4+ T cells and the cancer cells and promotion of the co-stimulation of the CD4+ T cells, thereby producing the CD4+ CTLs. In some embodiments, provided methods further comprise contacting with IL-2.

In some embodiments, provided are methods of producing CD4+ CTLs that comprise contacting CD4+ T cells with one or more populations of cancer cells that present an antigen by a class II MHC molecule and one or more soluble agents that promote co-stimulation of cells mediated by (i) CD27/CD70 signaling and/or (ii) OX40L/OX40 signaling, under conditions and for a time suitable for the formation of at least one immune complex between the CD4+ T cells and the cancer cells and promotion of the co-stimulation of the CD4+ T cells, thereby producing the CD4+ CTLs. In some embodiments, one or more soluble agents comprise an agonistic antibody. In some embodiments, one or more soluble agents comprise a fusion protein (e.g., an Fc fusion protein). In some embodiments, provided methods further comprise contacting with IL-2.

In some embodiments, CD4T cells for use in producing CD4+ CTLs by methods described herein are CD4+ T cells are naïve CD4+ T cells, memory CD4+ T cells, central memory CD4+ T cells, effector memory CD4+ T cells, Trp1-specific CD4+ T cells, tumor antigen-specific CD4+ T cells, neoantigen-specific CD4+ T cells, CD4+ T cells derived from peripheral blood mononuclear cells (PBMCs), CD4+ T cells derived from cord blood, CD4+ T cells derived from tumor-infiltrating lymphocytes, primary CD4+ T cells, CD4+ T cells derived from a cell line, CD4+ cells expanded in vitro, and/or CD4+ cells activated in vitro.

In some embodiments, CD4+ T cells are allogeneic, syngeneic, or autologous to the APCs. In some embodiments, CD4+ T cells are allogeneic, syngeneic, or autologous to the MHC II-expressing cancer cells.

In some embodiments, CD4+ T cells are selected from the group consisting of mammalian CD4+ T cells, rodent CD4+ T cells, mouse CD4+ T cells, primate CD4+ T cells, and human CD4+ T cells.

### CD4+ CTLs

In some embodiments, the present disclosure provides CD4+ CTLs produced by methods described herein.

In some embodiments, CD4+ CTLs provided herein express a granzyme, such as, e.g., granzyme B. In some embodiments, CD4+ CTLs produced by methods provided herein express a perforin. In some embodiments, CD4+ CTLs produced by methods provided herein express a granzyme and a perforin. In some embodiments, CD4+ CTLs provided herein express CD69. In some embodiments, CD4+ CTLs provided herein express a granzyme (e.g., granzyme B), a perforin, and CD69. In some embodiments, CD4+ CTLs provided herein are Eomes-programmed.

In some embodiments, CD4+ CTLs provided herein secrete cytokine. In some embodiments, CD4+ CTLs secrete IFNγ.

In some embodiments, CD4+ CTLs provided herein induce an antigen-specific T cell immune response. In some embodiments, CD4+ CTLs can target multiple tumor antigens. In some embodiments, CD4+ CTLs provided herein induce a multi-antigen specific T cell immune response.

In some embodiments, CD4+ CTLs provided herein are effectively used as a monotherapy for treating cancer.

In some embodiments, CD4+ CTLs provided herein are capable of killing cancer cells. In some embodiments, CD4+ CTLs are capable of directly killing MHC-II⁺ cancer cells. In some embodiments, CD4+ CTLs provided herein directly kill cancer cells that express on their cell surface the MHC II-antigen complex specifically recognized by the CD4+ CTLs.

In some embodiments, CD4+ CTLs provided herein proliferate. In some embodiments, CD4+ CTLs provided herein can be expanded in vitro and/or ex vivo.

In some embodiments, CD4+ CTLs provide long-lasting tumor immunity. In some embodiments, CD4+ CTLs provided herein persist in vivo. In some embodiments, CD4+ CTLs provided herein persist in vivo for at least 6 months, at least 7 months, at least 8 months, at least 9 months, at least 10 months, at least 11 months, at least 1 year, at least 1.5 years, at least 2 years, at least 3 years, at least 4 years, at least 5 years, at least 6 years, at least 7 years, at least 8 years, at least 9 years, or at least 10 years. In some embodiments, CD4+ CTLs provided herein persist in vivo for a duration that is between 6 months to 10 years, or any range in between inclusive, such as 6 months to 12 months, 6 months to 2 years, 6 months to 5 years, and the like.

In some embodiments, CD4+ CTLs provided herein execute CD4+ helper function. In some embodiments, CD4+ helper function comprises (i) licensing APC to prime anti-cancer CD8+ CTLs (e.g., secreting cytokine, activating APC, and/or recruiting APC), (ii) activating anti-cancer NK cells, (iii) activating anti-cancer macrophages, and/or (iv) activating anti-cancer immune cells.

CD4+ CTLs produced by methods described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells, or by detecting active caspase-3 in target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shigii (eds.) (1979) Selected Methods in Cellular Immunology, Freeman Publishing, San Francisco, CA; Green and Reed (1998) Science 281:1309, and references cited therein.

Cytotoxicity assays for determining CTL activity may be performed using any one of several techniques and methods routinely practiced in the art (e.g., Henkart el al., “Cytotoxic T-Lymphocytes” in Fundamental Immunology, Paul (ed.) (2003 Lippincott Williams & Wilkins, Philadelphia, PA), pages 1127-50, and references cited therein).

In some embodiments, CD4+ CTLs provided herein have MHC-II-restricted perforin/granzyme-mediated cytotoxicity. In some embodiments, CD4+ CTLs provided herein are characterized by expression of certain markers, including, for example, CD69, perforin, and/or granzyme, such as granzyme B. In some embodiments, CD4+ CTL production is dependent on the transcription factor, eomesodermin (Eomes) (i.e., Eomes-programmed).

In some embodiments, CD4+ CTLs generated by methods of the present disclosure are able to kill unmodified cells (e.g., unmodified tumor cells) that express the antigens that are recognized by TCRs expressed by the CD4+ CTLs when presented in the context of an MHC II molecule. In some embodiments, CD4+ CTLs generated by methods of the present disclosure can be used for adoptive cell therapy.

### Co-Stimulatory Agents

Productive activation of CD4+ T cells (e.g., naïve CD4+ T cells) requires two signals: signal 1 through TCR recognition of an antigen presented on the MHC complex, and signal 2 via a co-stimulatory pathway. The present disclosure provides the insight that tumor B cells (which naturally present endogenous antigens on MHC-II, enabling signal1) can be used to elicit tumor antigen-specific CD4+ CTLs, if co-stimulation capable of driving CD4+ CTL differentiation (signal 2) is also provided.

The term “immune co-stimulatory molecule” or “co-stimulatory agent” refers to a molecule that provides a non-antigen-specific signal for T cell proliferation and functional differentiation. Representative immune co-stimulatory molecules include, but are not limited to, CD80/B7-1, CD86/B7-2, CD70, CD27, OX40 ligand, OX40, 4-1BB ligand, 4-1BB, and GITR. Provided methods encompass the insight that co-stimulation through CD27-mediated signaling (e.g., CD70/CD27 signaling) and/or OX40-mediated signaling (e.g., OX40L/OX40 signaling) can promote activation and differentiation of CD4+ T cells into CD4+ CTLs. In some embodiments, co-stimulation is mediated through CD27-mediated signaling (e.g., CD70/CD27 signaling). In some embodiments, co-stimulation is through OX40-mediated signaling (e.g., OX40L/OX40 signaling). In some embodiments, co-stimulation is mediated through both CD27-mediated signaling (e.g., CD70/CD27 signaling) and OX40-mediated signaling (e.g., OX40L/OX40 signaling).

The present disclosure provides the additional insight that CD27-mediated co-stimulation (such as co-stimulation through CD70/CD27 signaling) is particularly useful for driving CD4+ CTL differentiation. In some embodiments, co-stimulation to activate CD4+ CTL differentiation is through both CD27-mediated co-stimulation (such as co-stimulation through CD70/CD27 signaling) and OX40-mediated co-stimulation (such as co-stimulation through OX40L/OX40 signaling).

Representative, non-limiting examples of nucleic acid and amino acid sequences of exemplary co-stimulatory molecules are provided in Table 1.

In some embodiments, a co-stimulatory agent is encoded by a nucleic acid sequence having at least 80%, at least 85%, at least 90%, at least 95%, at least 96%, at least 97%, at least 98%, or at least 99% identity to a nucleic acid sequence described herein (e.g., listed in Table 1). In some embodiments, a co-stimulatory agent is encoded by a nucleic acid sequence described herein (e.g., as set forth in Table 1).

In some embodiments, a co-stimulatory agent comprises an amino acid sequence having at least 80%, at least 85%, at least 90%, at least 95%, at least 96%, at least 97%, at least 98%, or at least 99% identity to an amino acid sequence described herein (e.g., listed in Table 1). In some embodiments, a co-stimulatory agent comprises an amino acid sequence described herein (e.g., as set forth in Table 1).

In some embodiments, a co-stimulatory molecule is or comprises CD70 (also referred to as CD27L; LPFS3; CD27-L; CD27LG; TNFSF7; TNLG8A). CD70 is a member of the tumor necrosis factor (TNF) family of ligands, and functions to activate the co-stimulatory receptor CD27 to enhance T-cell-modulated immune responses.

In some embodiments, a CD27-activating co-stimulatory agent comprises a nucleic acid sequence encoding CD70. In some embodiments, a CD27-activating co-stimulatory agent comprises a CD70 amino acid sequence.

In some embodiments, a CD70 sequence is from a human. Exemplary human CD70 nucleic acid and amino acid sequences are available to the public at the GenBank database, for example, under NM_001252.5, NP_001243.1 and NM_001330332.2, NP 001317261.1. In some embodiments, a CD70 sequence is from a mouse. Exemplary murine CD70 nucleic acid and amino acid sequences are available to the public at the GenBank database, for example, under NM_011617.2 and NP_035747.1. Nucleic acid and polypeptide sequences of CD70 orthologs in other organisms are known in the art and any of these are also included within the scope of the disclosure.

In some embodiments, the nucleic acid sequence encoding CD70 has at least 80%, at least 85%, at least 90%, at least 95%, at least 96%, at least 97%, at least 98%, or at least 99% identity to any one of SEQ ID NOs: 2, 4, and 6. In some embodiments, the nucleic acid sequence encoding CD70 comprises a sequence as set forth in any one of SEQ ID NOs: 2, 4, and 6.

In some embodiments, the CD70 amino acid sequence has at least 80%, at least 85%, at least 90%, at least 95%, at least 96%, at least 97%, at least 98%, or at least 99% identity to any one of SEQ ID NOs: 1, 3, and 5. In some embodiments, the CD70 amino acid sequence comprises a sequence as set forth in any one of SEQ ID NOs: 1, 3, and 5.

In some embodiments, a co-stimulatory molecule is or comprises CD27 (also referred to as S152, S152. LPFS2, T14, TNFRSF7, Tp55). CD27 is a 55 kDa type I transmembrane protein in the tumor necrosis factor receptor (TNFR) family that co-stimulates T-cell activation after binding to its ligand CD70. In humans, CD27 is constitutively expressed by I T cells and also expressed (e.g., transiently upregulated) on activated T cells.

In some embodiments, a CD27-activating co-stimulatory agent comprises a nucleic acid sequence encoding CD27. In some embodiments, a CD27-activating co-stimulatory agent comprises a CD27 amino acid sequence. In some embodiments, a CD27 is from a human. Exemplary human CD27 nucleic acid and amino acid sequences are available to the public at the GenBank database under NM_001242.5, NP_001233.2, NM_001413263.1, NP_001400192.1, NM_001413264.1, NP_001400193.1, NM_001413265.1, and NP_001400194.1. In some embodiments, a CD27 sequence is from a mouse. Exemplary murine CD27 nucleic acid and amino acid sequences are available to the public at the GenBank database, for example, under NM_001033126.2, NP_001028298.1, NM_001042564.1, NP_001036029.1, NM_001286753.1, and NP_001273682.1. In addition, nucleic acid and polypeptide sequences of CD27 orthologs in other organisms are known in the art.

In some embodiments, a nucleic acid sequence encoding CD27 has at least 80%, at least 85%, at least 90%, at least 95%, at least 96%, at least 97%, at least 98%, or at least 99% identity SEQ ID NO: 8. In some embodiments, a nucleic acid sequence encoding CD27 comprises a sequence as set forth in SEQ ID NO: 8.

In some embodiments, a CD27 amino acid sequence has at least 80%, at least 85%, at least 90%, at least 95%, at least 96%, at least 97%, at least 98%, or at least 99% identity to SEQ ID NO: 7. In some embodiments, a CD27 amino acid sequence comprises a sequence as set forth in SEQ ID NO: 7.

In some embodiments, a co-stimulatory molecule is or comprises OX40L (also referred to as TNFSF4, CD134L, CD252, GP34, OX-40L, OX4OL, TNLG2B, TXGP1). In some embodiments, an OX40-activating co-stimulatory agent comprises a nucleic acid sequence encoding OX40L. In some embodiments, an OX40-activating co-stimulatory agent comprises an OX40L amino acid sequence.

OX40L is predominantly expressed in antigen presenting cells. Exemplary OX40L nucleic acid and amino acid sequences are available to the public at the GenBank database. In some embodiments, an OX40L is from a human. Exemplary human OX40L nucleic acid and amino acid sequences are available to the public at the GenBank database, for example, under NM_001297562.2, NP_001284491.1, NM_003326.5, NP_003317.1.

In some embodiments, a OX40L sequence is from a mouse. Exemplary murine OX40L nucleic acid and amino acid sequences are available to the public at the GenBank database, for example, under NM_009452.2 and NP_033478.1. In addition, nucleic acid and polypeptide sequences of OX40L orthologs in other organisms are known in the art and any of these are also included within the scope of the disclosure.

In some embodiments, a nucleic acid sequence encoding OX40L has at least 80%, at least 85%, at least 90%, at least 95%, at least 96%, at least 97%, at least 98%, or at least 99% to SEQ ID NO: 10. In some embodiments, an OX40L nucleic acid sequence comprises a sequence as set forth in SEQ ID NO: 10.

In some embodiments, an OX40L amino acid sequence has at least 80%, at least 85%, at least 90%, at least 95%, at least 96%, at least 97%, at least 98%, or at least 99% identity to SEQ ID NO: 9. In some embodiments, an OX40L amino acid sequence comprises a sequence as set forth in SEQ ID NO: 9.

In some embodiments, a co-stimulatory molecule is or comprises OX40 (also referred to as TNFRSF4, ACT35; CD134; IMD16; TXGP1L). OX40 is not constitutively expressed in untreated T cells but is induced after participation of T cell receptors (TCRs). OX40 expression is induced in activated CD4+ T cells, but is generally not expressed by resting naïve T cells or most resting memory T cells.

In some embodiments, an OX40-activating co-stimulatory agent comprises a nucleic acid sequence encoding OX40. In some embodiments, an OX40-activating co-stimulatory agent comprises an OX40 amino acid sequence.

Exemplary OX40 nucleic acid and amino acid sequences are available to the public at the GenBank database. In some embodiments, an OX40 is from a human.

Exemplary human OX40L nucleic acid and amino acid sequences are available to the public at the GenBank database, for example, under NM_001410709.1, NP_001397638.1, NM_003327.4, NP_003318.1. In some embodiments, a OX40L sequence is from a mouse. Exemplary murine OX40 nucleic acid and amino acid sequences are available to the public at the GenBank database, for example, under NM_011659.2 and NP_035789.1. In addition, nucleic acid and polypeptide sequences of OX40 orthologs in other organisms are known in the art and any of these are also included within the scope of the disclosure.

In some embodiments, a nucleic acid sequence encoding OX40 has at least 80%, at least 85%, at least 90%, at least 95%, at least 96%, at least 97%, at least 98%, or at least 99% to SEQ ID NO: 12. In some embodiments, an OX40 nucleic acid sequence comprises a sequence as set forth in SEQ ID NO: 12.

In some embodiments, an OX40 amino acid sequence has at least 80%, at least 85%, at least 90%, at least 95%, at least 96%, at least 97%, at least 98%, or at least 99% identity to SEQ ID NO: 11. In some embodiments, an OX40 amino acid sequence comprises a sequence as set forth in SEQ ID NO: 11.

TABLE 1

| Description | Sequence |
|---|---|
| SEQ ID NO: 1 | MPEEGRPCPWVRWSGTAFQRQWPWLLLVVFITVFCCWFHCSGLLSKQQQRLLEHPEPHT |
| Exemplary CD70 | AELQLNLTVPRKDPTLRWGAGPALGRSFTHGPELEEGHLRIHQDGLYRLHIQVTLANCS |
| amino acid | SPGSTLQHRATLAVGICSPAAHGISLLRGRFGQDCTVALQRLTYLVHGDVLCTNLTLPL |
| NP_035747.1 | LPSRNADETFFGVQWICP |
| [Mus musculus] | |
| SEQ ID NO: 2 | GAAGGTGCCA AAAGCTCCAG GGGATTTCCC TGCCCTCCGA GAAGAGGCCC |
| Exemplary CD70 | AGTTCTTCCC CTGCATCGGA CATCCCCGAG GTTCTAAGGG CAGGTCAAGG |
| nucleic acid | CAGGCAGAAG CTTCAAAAGC TCGGCTGAGG AGGCTACAGC TTCCCGCTGC |
| NM 011617.2 | CTTCAGGCCG CTGCTTCCGT GCAGGGATGC CGGAGGAAGG TCGCCCTTGC |
| [Mus musculus] | CCCTGGGTTC GCTGGAGCGG GACCGCGTTC CAGCGCCAAT GGCCATGGCT |
| | GCTGCTGGTG GTGTTTATTA CTGTGTTTTG CTGTTGGTTT CATTGTAGCG |
| | GACTACTCAG TAAGCAGCAA CAGAGGCTGC TGGAGCACCC TGAGCCGCAC |
| | ACAGCTGAGT TACAGCTGAA TCTCACAGTT CCTCGGAAGG ACCCCACACT |
| | GCGCTGGGGA GCAGGCCCAG CCTTGGGAAG GTCCTTCACA CACGGACCAG |
| | AGCTGGAGGA GGGCCATCTG CGTATCCATC AAGATGGCCT CTACAGGCTG |
| | CATATCCAGG TGACACTGGC CAACTGCTCT TCCCCAGGCA GCACCCTGCA |
| | GCACAGGGCC ACCCTGGCTG TGGGCATCTG CTCCCCCGCT GCGCACGGCA |
| | TCAGCTTGCT GCGTGGGCGC TTTGGACAGG ACTGTACAGT GGCATTACAG |
| | CGCCTGACAT ACCTGGTCCA CGGAGATGTC CTCTGTACCA ACCTCACCCT |
| | GCCTCTGCTG CCGTCCCGCA ACGCTGATGA GACCTTCTTT GGAGTTCAGT |
| | GGATATGCCC TTGACCACAA CTCCAGGATG ACTTGTGAAT ATTTTTTTTC |
| | TTTTCAAGTT CTACGTATTT ATAAATGTAT ATAGTACACA TA |
| SEQ ID NO: 3 | MPEEGSGCSV RRRPYGCVLR AALVPLVAGL VICLVVCIQR FAQAQQQLPL |
| Exemplary CD70 | ESLGWDVAEL QLNHTGPQQD PRLYWQGGPA LGRSFLHGPE LDKGQLRIHR |
| amino acid | DGIYMVHIQV TLAICSSTTA SRHHPTTLAV GICSPASRSI SLLRLSFHQG |
| sequence | CTIASQRLTP LARGDTLCTN LTGTLLPSRN TDETFFGVQW VRP |
| Isoform 1 | |
| NP_001243.1 | |
| SEQ ID NO: 4 | AGAGAGGGGC AGGCTGGTCC CCTGACAGGT TGAAGCAAGT AGACGCCCAG |
| Exemplary CD70 | GAGCCCCGGG AGGGGGCTGC AGTTTCCTTC CTTCCTTCTC GGCAGCGCTC |
| nucleotide | CGCGCCCCCA TCGCCCCTCC TGCGCTAGCG GAGGTGATCG CCGCGGCGAT |
| sequence | GCCGGAGGAG GGTTCGGGCT GCTCGGTGCG GCGCAGGCCC TATGGGTGCG |
| Isoform 1 | TCCTGCGGGC TGCTTTGGTC CCATTGGTCG CGGGCTTGGT GATCTGCCTC |
| NM_001252.5 | GTGGTGTGCA TCCAGCGCTT CGCACAGGCT CAGCAGCAGC TGCCGCTCGA |
| | GTCACTTGGG TGGGACGTAG CTGAGCTGCA GCTGAATCAC ACAGGACCTC |
| | AGCAGGACCC CAGGCTATAC TGGCAGGGGG GCCCAGCACT GGGCCGCTCC |
| | TTCCTGCATG GACCAGAGCT GGACAAGGGG CAGCTACGTA TCCATCGTGA |
| | TGGCATCTAC ATGGTACACA TCCAGGTGAC GCTGGCCATC TGCTCCTCCA |
| | CGACGGCCTC CAGGCACCAC CCCACCACCC TGGCCGTGGG AATCTGCTCT |
| | CCCGCCTCCC GTAGCATCAG CCTGCTGCGT CTCAGCTTCC ACCAAGGTTG |
| | TACCATTGCC TCCCAGCGCC TGACGCCCCT GGCCCGAGGG GACACACTCT |
| | GCACCAACCT CACTGGGACA CTTTTGCCTT CCCGAAACAC TGATGAGACC |
| | TTCTTTGGAG TGCAGTGGGT GCGCCCCTGA CCACTGCTGC TGATTAGGGT |
| | TTTTTAAATT TTATTTTATT TTATTTAAGT TCAAGAGAAA AAGTGTACAC |
| | ACAGGGGCCA CCCGGGGTTG GGGTGGGAGT GTGGTGGGGG GTAGTGGTGG |
| | CAGGACAAGA GAAGGCATTG AGCTTTTTCT TTCATTTTCC TATTAAAAAA |
| | TACAAAAATC A |
| SEQ ID NO: 5 | MPEEGSGCSV RRRPYGCVLR AALVPLVAGL VICLVVCIQR FAQAQQQLPL |
| Exemplary CD70 | ESLGWDVAEL QLNHTGPQQD PRLYWQGGPA LGRSFLHGPE LDKGQLRIHR |
| amino acid | DGIYMVHIQV TLAICSSTTA SRHHPTTLAV GICSPASRSI SLLRLSFHQG |
| sequence | LFGFWNWGLK VKCFLRHLIW TAHCFIPLTQ LVFMQALQSW RNHHCSHFTD |
| Isoform 2 | EENRGVNR |
| NP_001317261.1 | |
| SEQ ID NO: 6 | AGAGAGGGGC AGGCTGGTCC CCTGACAGGT TGAAGCAAGT AGACGCCCAG |
| Exemplary CD70 | GAGCCCCGGG AGGGGGCTGC AGTTTCCTTC CTTCCTTCTC GGCAGCGCTC |
| nucleotide | CGCGCCCCCA TCGCCCCTCC TGCGCTAGCG GAGGTGATCG CCGCGGCGAT |
| sequence | GCCGGAGGAG GGTTCGGGCT GCTCGGTGCG GCGCAGGCCC TATGGGTGCG |
| Isoform 2 | TCCTGCGGGC TGCTTTGGTC CCATTGGTCG CGGGCTTGGT GATCTGCCTC |
| NM_001330332.2 | GTGGTGTGCA TCCAGCGCTT CGCACAGGCT CAGCAGCAGC TGCCGCTCGA |
| | GTCACTTGGG TGGGACGTAG CTGAGCTGCA GCTGAATCAC ACAGGACCTC |
| | AGCAGGACCC CAGGCTATAC TGGCAGGGGG GCCCAGCACT GGGCCGCTCC |
| | TTCCTGCATG GACCAGAGCT GGACAAGGGG CAGCTACGTA TCCATCGTGA |
| | TGGCATCTAC ATGGTACACA TCCAGGTGAC GCTGGCCATC TGCTCCTCCA |
| | CGACGGCCTC CAGGCACCAC CCCACCACCC TGGCCGTGGG AATCTGCTCT |
| | CCCGCCTCCC GTAGCATCAG CCTGCTGCGT CTCAGCTTCC ACCAAGGGCT |
| | TTTTGGATTT TGGAACTGGG GACTCAAAGT CAAGTGCTTC TTACGGCATT |
| | TAATATGGAC TGCACACTGT TTTATCCCAT TAACTCAGCT CGTGTTCATG |
| | CAAGCCCTAC AAAGCTGGAG GAATCATCAT TGTTCCCATT TCACAGATGA |
| | GGAAAACAGA GGCGTAAACC GTTGAGTCAT CTGTCCAAAG TTACTGGTTT |
| | TGTAACAGCT GGAACCTCAG TATTTAACAG TTGAGGAAAA TAGTGGCAGA |
| | AAACCTGAGT CACAAGCCCG AGGCAATTCA TCTACATAGT AGTGGACTCA |
| | GGATTCCAAG CCAGATAATC CCGCATCTTG CATCCATGAT CTCACTTCTA |
| | GAAGGGGAAG TGAGAGGAGT GGTGGGGTGT CCCAGCTGAG GGAGGGAAAA |
| | TGAGACCCAT GCGCCACCCG CCCTCACAAA TCTAGCCCTG CAGCCCCTCT |
| | CCAACCTTAC CTGGACTCAG AACCATGCAT CTGGAAAAGC CACAGACACT |
| | CGACGCCAGC CTGTGAAAGC AGCCGGGAGG GAGGCTGTAT CCTGCAAAGC |
| | CACAGGGAAG GGGCTGCCCA AGACCATGGG AACCCACCAC TTACATCAGC |
| | GTGACCTGGA TGTGAGACAC AGAGTCAAAG GAGATTATCT GGCAGCTTTA |
| | AGATTGGACT GCCTGACTAT TCACAATAGC AAAGACTTGG AACCAACCCA |
| | AATGTCCATC AATGATAGAC TGGATTAAGA AAATGTGGCA CATATACACC |
| | ATGGAATACT ATGCAGCCAT AAAAACGGAT GAGTTCATGT CCTTCATAGG |
| | GACATGGATG AAGCTGGAAA CCATCATTCT GAGCAAACTA TTGCAAGGAC |
| | AGAAAACCAA ACACCGCATG TTCTCACTCA TAGGTGGAAA TTGAACAATG |
| | AGAATACTTG GACACAGGGA GGAGAACATC ACACACCGGG GCCTGTCGTG |
| | GAGTGTGGAG TCGGGGGAGG GGGGGAGGGA TAGCATTAGG AGAAATACCT |
| | AATGTAAATG ACAAATTAAT GGGTGCAGTA AACCAACACG GCACATATAT |
| | ACATATGTAA CAAATCTGCA CGTTGTGCAC ATGTACCCTA GAACTTAAAG |
| | TATAATTAAA AAAAAAAAAA AAAAAAGATT TGACTGCCTG GCCAGGCACA |
| | GTGGCTCATG CCTATAATCC TAACACTTTG GGAGGCCGAG GCGAGCAGAT |
| | TGCCTGAGCT GAAGAGTTCG AGACCAGCCT GGGCAATATG GTGAAACCCC |
| | GTTTCTACTA AAATACAAAA AAAAAAAAAT TAGCCAGGCG TGGCAGCGTG |
| | CGCCTGTAGT CCCCGCGACT TGGAGGCTGA TGCAGGAGAA TCGCTTTAAC |
| | CCGGGAGGCA GAGGTTGCAG TGAGCCGAGA TCGAGCCACT GTACTCCAGA |
| | CTGGGTGACA GAGCGACACT CTGTCTCAAA AAAAAAAAAA AGATTTAACT |
| | GCCCCACTGG ATTTTGGAAT TGCAGGGGAC CTTCAGCCCC TTCGTTTGTC |
| | CAGTTTCTCC CATTTGGAAT GGGTATATCC AACGCCTGTA CCCCATTGTA |
| | TATAGGAAGT AACTAACTTG CTTTTGATTT TACAGGCTCA CAGAAGGGAC |
| | GTGCCTTGTC TCAGATGAGA TTTTGGACTG TGGACTTTTG AGTTAATGCT |
| | GAAATGAGTT AAGACTTTGG GGGATAGTTA GGAAGGCATG ACTAGTTTGA |
| | GGACATTATA CTCAATGAAA TAAGCCAGTC AAAAAGGAAA AATATGTATT |
| | GTATAAACTA ACTTACATTA ATCTACCACA TAGGATTAAA ACTGTTCACA |
| | GGAATATAAA ATAGAATTTT TTAGATA |
| SEQ ID NO: 7 | MARPHPWWLC VLGTLVGLSA TPAPKSCPER HYWAQGKLCC QMCEPGTFLV |
| Exemplary CD27 | KDCDQHRKAA QCDPCIPGVS FSPDHHTRPH CESCRHCNSG LLVRNCTITA |
| amino acid | NAECACRNGW QCRDKECTEC DPLPNPSLTA RSSQALSPHP QPTHLPYVSE |
| sequence | MLEARTAGHM QTLADFRQLP ARTLSTHWPP QRSLCSSDFI RILVIFSGMF |
| NP_001233.2 | LVFTLAGALF LHQRRKYRSN KGESPVEPAE PCHYSCPREE EGSTIPIQED |
| | YRKPEPACSP |
| SEQ ID NO : 8 | CTTCAAAGGT TGGCTTGCCA CCTGAAGCAG CCACTGCCCA GGGGGTGCAA |
| Exemplary CD27 | AGAAGAGACA GCAGCGCCCA GCTTGGAGGT GCTAACTCCA GAGGCCAGCA |
| nucleotide | TCAGCAACTG GGCACAGAAA GGAGCCGCCT GGGCAGGGAC CATGGCACGG |
| sequence | CCACATCCCT GGTGGCTGTG CGTTCTGGGG ACCCTGGTGG GGCTCTCAGC |
| NM_001242.5 | TACTCCAGCC CCCAAGAGCT GCCCAGAGAG GCACTACTGG GCTCAGGGAA |
| | AGCTGTGCTG CCAGATGTGT GAGCCAGGAA CATTCCTCGT GAAGGACTGT |
| | GACCAGCATA GAAAGGCTGC TCAGTGTGAT CCTTGCATAC CGGGGGTCTC |
| | CTTCTCTCCT GACCACCACA CCCGGCCCCA CTGTGAGAGC TGTCGGCACT |
| | GTAACTCTGG TCTTCTCGTT CGCAACTGCA CCATCACTGC CAATGCTGAG |
| | TGTGCCTGTC GCAATGGCTG GCAGTGCAGG GACAAGGAGT GCACCGAGTG |
| | TGATCCTCTT CCAAACCCTT CGCTGACCGC TCGGTCGTCT CAGGCCCTGA |
| | GCCCACACCC TCAGCCCACC CACTTACCTT ATGTCAGTGA GATGCTGGAG |
| | GCCAGGACAG CTGGGCACAT GCAGACTCTG GCTGACTTCA GGCAGCTGCC |
| | TGCCCGGACT CTCTCTACCC ACTGGCCACC CCAAAGATCC CTGTGCAGCT |
| | CCGATTTTAT TCGCATCCTT GTGATCTTCT CTGGAATGTT CCTTGTTTTC |
| | ACCCTGGCCG GGGCCCTGTT CCTCCATCAA CGAAGGAAAT ATAGATCAAA |
| | CAAAGGAGAA AGTCCTGTGG AGCCTGCAGA GCCTTGTCAT TACAGCTGCC |
| | CCAGGGAGGA GGAGGGCAGC ACCATCCCCA TCCAGGAGGA TTACCGAAAA |
| | CCGGAGCCTG CCTGCTCCCC CTGAGCCAGC ACCTGCGGGA GCTGCACTAC |
| | AGCCCTGGCC TCCACCCCCA CCCCGCCGAC CATCCAAGGG AGAGTGAGAC |
| | CTGGCAGCCA CAACTGCAGT CCCATCCTCT TGTCAGGGCC CTTTCCTGTG |
| | TACACGTGAC AGAGTGCCTT TTCGAGACTG GCAGGGACGA GGACAAATAT |
| | GGATGAGGTG GAGAGTGGGA AGCAGGAGCC CAGCCAGCTG CGCCTGCGCT |
| | GCAGGAGGGC GGGGGCTCTG GTTGTAAAAC ACACTTCCTG CTGCGAAAGA |
| | CCCACATGCT ACAAGACGGG CAAAATAAAG TGACAGATGA CCACC |
| SEQ ID NO : 9 | MVSHRYPRIQ SIKVQFTEYK KEKGFILTSQ KEDEIMKVQN NSVIINCDGF |
| Exemplary OX40L | YLISLKGYFS QEVNISLHYQ KDEEPLFOLK KVRSVNSLMV ASLTYKDKVY |
| amino acid | LNVTTDNTSL DDFHVNGGEL ILIHQNPGEF CVL |
| sequence | |
| NP_001284491.1 | |
| SEQ ID NO:10 | AAGGCTAACT GATTGCACAA CGGCTCTGAG CAGGAGAGAG CAAAAGGTGA |
| Exemplary OX40L | ACCACAGTTG TACAGCTTGC TGGTGGTTGG GGTCACATTG GACTATCAAT |
| nucleotide | ATCCAGGAGA TGTTGAGAAA AAGAGGGGAT CCTTTCCAAC AAGTCATCCA |
| sequence | GCAACTCTGG GACAGAAGGA AAGCTGTGGG AAGAAAAGCA GGGTGCTGGT |
| NM_001297562.2 | TTCTAGGACA CTAAACCTCA TGGTATCACA TCGGTATCCT CGAATTCAAA |
| | GTATCAAAGT ACAATTTACC GAATATAAGA AGGAGAAAGG TTTCATCCTC |
| | ACTTCCCAAA AGGAGGATGA AATCATGAAG GTGCAGAACA ACTCAGTCAT |
| | CATCAACTGT GATGGGTTTT ATCTCATCTC CCTGAAGGGC TACTTCTCCC |
| | AGGAAGTCAA CATTAGCCTT CATTACCAGA AGGATGAGGA GCCCCTCTTC |
| | CAACTGAAGA AGGTCAGGTC TGTCAACTCC TTGATGGTGG CCTCTCTGAC |
| | TTACAAAGAC AAAGTCTACT TGAATGTGAC CACTGACAAT ACCTCCCTGG |
| | ATGACTTCCA TGTGAATGGC GGAGAACTGA TTCTTATCCA TCAAAATCCT |
| | GGTGAATTCT GTGTCCTTTG AGGGGCTGAT GGCAATATCT AAAACCAGGC |
| | ACCAGCATGA ACACCAAGCT GGGGGTGGAC AGGGCATGGA TTCTTCATTG |
| | CAAGTGAAGG AGCCTCCCAG CTCAGCCACG TGGGATGTGA CAAGAAGCAG |
| | ATCCTGGCCC TCCCGCCCCC ACCCCTCAGG GATATTTAAA ACTTATTTTA |
| | TATACCAGTT AATCTTATTT ATCCTTATAT TTTCTAAATT GCCTAGCCGT |
| | CACACCCCAA GATTGCCTTG AGCCTACTAG GCACCTTTGT GAGAAAGAAA |
| | AAATAGATGC CTCTTCTTCA AGATGCATTG TTTCTATTGG TCAGGCAATT |
| | GTCATAATAA ACTTATGTCA TTGAAAACGG TACCTGACTA CCATTTGCTG |
| | GAAATTTGAC ATGTGTGTGG CATTATCAAA ATGAAGAGGA GCAAGGAGTG |
| | AAGGAGTGGG GTTATGAATC TGCCAAAGGT GGTATGAACC AACCCCTGGA |
| | AGCCAAAGCG GCCTCTCCAA GGTTAAATTG ATTGCAGTTT GCATATTGCC |
| | TAAATTTAAA CTTTCTCATT TGGTGGGGGT TCAAAAGAAG AATCAGCTTG |
| | TGAAAAATCA GGACTTGAAG AGAGCCGTCT AAGAAATACC ACGTGCTTTT |
| | TTTCTTTACC ATTTTGCTTT CCCAGCCTCC AAACATAGTT AATAGAAATT |
| | TCCCTTCAAA GAACTGTCTG GGGATGTGAT GCTTTGAAAA ATCTAATCAG |
| | TGACTTAAGA GAGATTTTCT TGTATACAGG GAGAGTGAGA TAACTTATTG |
| | TGAAGGGTTA GCTTTACTGT ACAGGATAGC AGGGAACTGG ACATCTCAGG |
| | GTAAAAGTCA GTACGGATTT TAATAGCCTG GGGAGGAAAA CACATTCTTT |
| | GCCACAGACA GGCAAAGCAA CACATGCTCA TCCTCCTGCC TATGCTGAGA |
| | TACGCACTCA GCTCCATGTC TTGTACACAC AGAAACATTG CTGGTTTCAA |
| | GAAATGAGGT GATCCTATTA TCAAATTCAA TCTGATGTCA AATAGCACTA |
| | AGAAGTTATT GTGCCTTATG AAAAATAATG ATCTCTGTCT AGAAATACCA |
| | TAGACCATAT ATAGTCTCAC ATTGATAATT GAAACTAGAA GGGTCTATAA |
| | TCAGCCTATG CCAGGGCTTC AATGGAATAG TATCCCCTTA TGTTTAGTTG |
| | AAATGTCCCC TTAACTTGAT ATAATGTGTT ATGCTTATGG CGCTGTGGAC |
| | AATCTGATTT TTCATGTCAA CTTTCCAGAT GATTTGTAAC TTCTCTGTGC |
| | CAAACCTTTT ATAAACATAA ATTTTTGAGA TATGTATTTT AAAATTGTAG |
| | CACATGTTTC CCTGACATTT TCAATAGAGG ATACAACATC ACAGAATCTT |
| | TCTGGATGAT TCTGTGTTAT CAAGGAATTG TACTGTGCTA CAATTATCTC |
| | TAGAATCTCC AGAAAGGTGG AGGGCTGTTC GCCCTTACAC TAAATGGTCT |
| | CAGTTGGATT TTTTTTTCCT GTTTTCTATT TCCTCTTAAG TACACCTTCA |
| | ACTATATTCC CATCCCTCTA TTTTAATCTG TTATGAAGGA AGGTAAATAA |
| | AAATGCTAAA TAGAAGAAAT TGTAGGTAAG GTAAGAGGAA TCAAGTTCTG |
| | AGTGGCTGCC AAGGCACTCA CAGAATCATA ATCATGGCTA AATATTTATG |
| | GAGGGCCTAC TGTGGACCAG GCACTGGGCT AAATACTTAC ATTTACAAGA |
| | ATCATTCTGA GACAGATATT CAATGATATC TGGCTTCACT ACTCAGAAGA |
| | TTGTGTGTGT GTTTGTGTGT GTGTGTGTGT GTGTATTTCA CTTTTTGTTA |
| | TTGACCATGT TCTGCAAAAT TGCAGTTACT CAGTGAGTGA TATCCGAAAA |
| | AGTAAACGTT TATGACTATA GGTAATATTT AAGAAAATGC ATGGTTCATT |
| | TTTAAGTTTG GAATTTTTAT CTATATTTCT CACAGATGTG CAGTGCACAT |
| | GCAGGCCTAA GTATATGTTG TGTGTGTTGT TTGTCTTTGA TGTCATGGTC |
| | CCCTCTCTTA GGTGCTCACT CGCTTTGGGT GCACCTGGCC TGCTCTTCCC |
| | ATGTTGGCCT CTGCAACCAC ACAGGGATAT TTCTGCTATG CACCAGCCTC |
| | ACTCCACCTT CCTTCCATCA AAAATATGTG TGTGTGTCTC AGTCCCTGTA |
| | AGTCATGTCC TTCACAGGGA GAATTAACCC TTCGATATAC ATGGCAGAGT |
| | TTTGTGGGAA AAGAATTGAA TGAAAAGTCA GGAGATCAGA ATTTTAAATT |
| | TGACTTAGCC ACTAACTAGC CATGTAACCT TGGGAAAGTC ATTTCCCATT |
| | TCTGGGTCTT GCTTTTCTTT CTGTTAAATG AGAGGAATGT TAAATATCTA |
| | ACAGTTTAGA ATCTTATGCT TACAGTGTTA TCTGTGAATG CACATATTAA |
| | ATGTCTATGT TCTTGTTGCT ATGAGTCAAG GAGTGTAACC TTCTCCTTTA |
| | CTATGTTGAA TGTATTTTTT TCTGGACAAG CTTACATCTT CCTCAGCCAT |
| | CTTTGTGAGT CCTTCAAGAG CAGTTATCAA TTGTTAGTTA GATATTTTCT |
| | ATTTAGAGAA TGCTTAAGGG ATTCCAATCC CGATCCAAAT CATAATTTGT |
| | TCTTAAGTAT ACTGGGCAGG TCCCCTATTT TAAGTCATAA TTTTGTATTT |
| | AGTGCTTTCC TGGCTCTCAG AGAGTATTAA TATTGATATT AATAATATAG |
| | TTAATAGTAA TATTGCTATT TACATGGAAA CAAATAAAAG ATCTCAGAAT |
| | TCACTA |
| SEQ ID NO: 11 | MCVGARRLGR GPCAALLLLG LGLSTVTGLH CVGDTYPSND RCCHECRPGN |
| Exemplary OX40 | GMVSRCSRSQ NTVCRPCGPG FYNDVVSSKP CKPCTWCNLR SGSERKQLCT |
| amino acid | ATQDTVCRCR AGTQPLDSYK PGVDCAPCPP GHFSPGDNQA CKPWTNCTLA |
| sequence | GKHTLQPASN SSDAICEDRD PPATQPQETQ GPPARPITVQ PTEAWPRTSQ |
| NP_003318.1 | GPSTRPVEVP GGRAVAAILG LGLVLGLLGP LAILLALYLL RRDQRLPPDA |
| | HKPPGGGSFR TPIQEEQADA HSTLAKI |
| SEQ ID NO: 12 | AGACTCTGGC GACAGCAGAG ACGAGGATGT GCGTGGGGGC TCGGCGGCTG |
| Exemplary OX40 | GGCCGCGGGC CGTGTGCGGC TCTGCTCCTC CTGGGCCTGG GGCTGAGCAC |
| nucleotide | CGTGACGGGG CTCCACTGTG TCGGGGACAC CTACCCCAGC AACGACCGGT |
| sequence | GCTGCCACGA GTGCAGGCCA GGCAACGGGA TGGTGAGCCG CTGCAGCCGC |
| NM_003327.4 | TCCCAGAACA CGGTGTGCCG TCCGTGCGGG CCGGGCTTCT ACAACGACGT |
| | GGTCAGCTCC AAGCCGTGCA AGCCCTGCAC GTGGTGTAAC CTCAGAAGTG |
| | GGAGTGAGCG GAAGCAGCTG TGCACGGCCA CACAGGACAC AGTCTGCCGC |
| | TGCCGGGCGG GCACCCAGCC CCTGGACAGC TACAAGCCTG GAGTTGACTG |
| | TGCCCCCTGC CCTCCAGGGC ACTTCTCCCC AGGCGACAAC CAGGCCTGCA |
| | AGCCCTGGAC CAACTGCACC TTGGCTGGGA AGCACACCCT GCAGCCGGCC |
| | AGCAATAGCT CGGACGCAAT CTGTGAGGAC AGGGACCCCC CAGCCACGCA |
| | GCCCCAGGAG ACCCAGGGCC CCCCGGCCAG GCCCATCACT GTCCAGCCCA |
| | CTGAAGCCTG GCCCAGAACC TCACAGGGAC CCTCCACCCG GCCCGTGGAG |
| | GTCCCCGGGG GCCGTGCGGT TGCCGCCATC CTGGGCCTGG GCCTGGTGCT |
| | GGGGCTGCTG GGCCCCCTGG CCATCCTGCT GGCCCTGTAC CTGCTCCGGA |
| | GGGACCAGAG GCTGCCCCCC GATGCCCACA AGCCCCCTGG GGGAGGCAGT |
| | TTCCGGACCC CCATCCAAGA GGAGCAGGCC GACGCCCACT CCACCCTGGC |
| | CAAGATCTGA CCTGGGCCCA CCAAGGTGGA CGCTGGGCCC CGCCAGGCTG |
| | GAGCCCGGAG GGTCTGCTGG GCGAGCAGGG CAGGTGCAGG CCGCCTGCCC |
| | CGCCACGCTC CTGGGCCAAC TCTGCACCGT TCTAGGTGCC GATGGCTGCC |
| | TCCGGCTCTC TGCTTACGTA TGCCATGCAT ACCTCCTGCC CCGCGGGACC |
| | ACAATAAAAA CCTTGGCAGA CGGGA |

Co-stimulation can be mediated by any method known in the art. In some embodiments, co-stimulation is mediated by agonistic antibodies that specifically bind an immune co-stimulatory molecule. In some embodiments, co-stimulation is mediated at least in part by an anti-CD27 agonist antibody and/or an anti-OX40 agonist antibody.

In some embodiments, one or more co-stimulatory agents comprise an anti-CD27 agonist antibody. Exemplary anti-CD27 agonist antibodies are known in the art. For example, WO 2012/004367 describes an anti-human agonist antibody designated hCD27.15 and WO 2011/130434 describes an anti-human CD27 antibody designated 1F5. In some embodiments, an anti-mouse CD27 antibody is BE0348, clone RM27-3E5 (BioXCell).

In some embodiments, one or more co-stimulatory agents comprise an anti-OX40 agonist antibody. Exemplary anti-OX40 agonist antibodies are known in the art, such as, for example, INCAGN01949, 9B12, MEDI0562, and BMS-986178. In some embodiments, an anti-mouse OX40 (CD134) antibody is BE0031, clone OX-86 (BioXCell).

In some embodiments, co-stimulation is mediated by a soluble fusion protein of an immune co-stimulatory molecule. In some embodiments, co-stimulation is mediated by a CD70 fusion protein (e.g., a CD70-Fc fusion protein) and/or an OX40L fusion protein (e.g., an OX40L-Fc fusion protein).

In some embodiments, co-stimulation is mediated by a CD70 fusion protein (e.g., a CD70-Fc fusion protein). In some embodiments, a CD70 fusion protein binds to its co-stimulatory surface receptor CD27 expressed on T lymphocytes. Exemplary CD70-Fc fusion proteins are commercially available: Ag28070 (Proteintech®) and CD70-Fc (SKU #: FCL2530, G&P Biosciences®).

In some embodiments, antigen-presenting cells (e.g., antigen presenting cancer cells) are supplemented with a CD27-activating agent (e.g., CD70, e.g., ectopically expressed CD70) and/or an OX40-activating agent (e.g., OX40L, e.g., ectopically expressed OX40L), to activate CD4+ T cells to form CD4+ CTLs.

### Antigen Presenting Cells

The term “antigen-presenting cell” is any of a variety of cells capable of displaying, acquiring, and/or presenting at least one antigen or antigenic fragment on its cell surface. In general, an antigen-presenting cell (APC) can be any cell that induces and/or enhances an immune response against an antigen or antigenic composition. Full activation of I T cells can be achieved by an antigen displayed by an APC in the form of a peptide bound to an MHC, which provides specificity to the response, and a co-stimulatory signal, which is antigen nonspecific and facilitates the development of an effective immune response of adaptive immunity. T cell co-stimulation increases T cell proliferation, differentiation and survival. Activation of T cells without co-stimulation may lead to T cell anergy, T cell deletion or the development of immune tolerance. Additional molecules expressed by the APC or other immune cells that may aid or enhance an immune response include secreted molecules, such as cytokines and cytotoxic molecules. Some cells, such as dendritic cells, are professional APCs. In some embodiments, APCs are derived from cancer cells. In some embodiments, APCs are engineered (e.g., cancer cells engineered to express MHC II, as well as CD70 and/or OX40L).

The term “MHC” refers to “major histocompatibility antigen.” In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such, the terms “MHC” and “HLA” are used interchangeably herein. The HLA system in humans has its equivalent in the mouse, i.e., the H2 system. The most studied HLA genes are the nine so-called classical MHC genes: HLA-A, HLA-B, HLA-C, HLA-DPA1, HLA-DPB 1, HLA-DQA1, HLA-DQB1, HLA-DRA, and HLA-DRB 1. In humans, the MHCs include at least three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 domains (alpha 1 and 2, and beta 1 and 2). Class I MHC molecules are expressed on virtually all nucleated cells. Peptide fragments presented in the context of class I MHC molecules are recognized by CD8+ T lymphocytes (traditionally called cytotoxic T lymphocytes or CTLs). CD8+ T lymphocytes frequently mature into cytotoxic effectors which can lyse cells bearing the stimulating antigen. Class II MHC molecules are expressed primarily on activated lymphocytes and professional APCs. CD4+ T lymphocytes (traditionally called helper T lymphocytes or HTLs) are activated with recognition of a unique peptide fragment presented by a class II MHC molecule, usually found on an APC, like a macrophage, dendritic cell or B cell. CD4+ T lymphocytes proliferate and secrete cytokines that either support an antibody-mediated response through the production of IL-4 or support a cell-mediated response through the production of IL-2 and IFN-gamma, or acquire direct killing activity (cytotoxicity).

In some embodiments, APCs (e.g., antigen presenting cancer cells) are or have been induced to express MHC II through contact with interferon gamma (IFNγ). Many cancers, including melanoma, bladder cancer, breast cancer, head and neck cancer, and hepatocellular carcinoma (HCC), are known to induce MHC II expression via IFNγ exposure, In some embodiments, APCs (e.g., antigen presenting cancer cells) are or have been induced to express MHCII by expression of class II transactivator (CIITA).

In some embodiments, APCs (e.g., antigen presenting cancer cells) endogenously (e.g., natively) express MHC-II. In some embodiments, APCs (e.g., antigen presenting cancer cells) are derived from a cancer that endogenously (e.g., natively) expresses MHC-II.

Any appropriate method may be used to transfect or transduce the cells, for example, APCs (e.g., cancer cells) are encompassed by methods described herein. Methods for delivering polynucleotides to host cells include, for example, use of cationic polymers, lipid-like molecules, and certain commercial products such as, for example, in vivo-jetPEI®. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

In some embodiments, APCs (e.g., antigen presenting cancer cells) are contacted with IL-2.

### Cancers

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

In some embodiments, a cancer is a B cell cancer. B-cell malignancies comprise a large collection of genetically, phenotypically, and clinically distinct diseases, including Hodgkin lymphoma (HL), most non-Hodgkin lymphomas (NHL), chronic lymphocytic leukemia (CLL), and B-cell acute lymphocytic leukemia (B-ALL). NHL can be further classified into many subtypes, including diffuse large B-cell lymphoma (DLBCL), follicular lymphoma, etc.

In some embodiments, CD4+ CTLs produced by methods of the current disclosure are suitable for killing and/or treating CD70-deficient cancers. In some embodiments, CD4+ CTL produced by methods of the current disclosure are suitable for killing and/or treating CD70-deficient B-cell tumors.

In some embodiments, CD4+ CTL produced by methods of the current disclosure are suitable for killing and/or treating CD70-deficient solid tumors.

In some embodiments, CD4+ CTL produced by methods of the current disclosure are suitable for killing and/or treating cancers with intact CD70.

In some embodiments, methods can produce CD4+ CTLs against a wide range of endogenous tumor antigens without the need to identify them in each patient.

In some embodiments, a cancer is a solid tumor. In some embodiments, a cancer is a melanoma, bladder cancer, breast cancer, head and neck cancer, and/or hepatocellular carcinoma (HCC), colon cancer, pancreatic cancer, kidney cancer, ovarian cancer, or brain cancer.

In some embodiments, a cancer presents at least one MHC II-antigen complex on its cell surface. In some embodiments, an antigen presented by an MHC-II-antigen complex is a tumor-associated antigen and/or a neoantigen.

In some embodiments, a cancer endogenously (e.g., natively) expresses CD70. In some embodiments, a cancer does not endogenously (e.g., does not natively) express CD70. For example, it is known that a subset of B cell cancers lose CD70 expression after initially expressing CD70. In some embodiments, cells derived from a cancer for use in provided methods has been engineered to exogenously express CD70.

In some embodiments, a cancer endogenously (e.g., natively) expresses OX40L. In some embodiments, a cancer does not endogenously (e.g., does not natively) express OX40L. In some embodiments, cells derived from a cancer for use in provided methods has been engineered to exogenously express OX40L.

### Nucleic Acids and Vectors

Provided herein are nucleic acid molecules useful in methods provided herein. For example, in some embodiments, provided are nucleic acid molecules that encode a co-stimulatory agent (e.g., an agent that promotes CD27-mediated and/or OX40-mediated co-stimulation). In some embodiments, provided are nucleic acid molecules that encode an agent that promotes CD27-mediated co-stimulation, such as wherein the agent is a CD70 polypeptide (e.g., fusion polypeptide, e.g., CD70-Fc) or anti-CD27 agonistic antibody. In some embodiments, provided are nucleic acid molecules that encode an agent that promotes OX40-mediated co-stimulation, such as wherein the agent is an OX40L polypeptide (e.g., fusion polypeptide, e.g., OX40L-Fc) or anti-OX40 agonistic antibody. In some embodiments, provided are nucleic acid molecules that encode an agent that promotes OX40-mediated co-stimulation, such as wherein the agent is an OX40L polypeptide or agonistic OX40 antibody. Methods for gene delivery into cells are well known in the art, are further described in the APC section above, and include, for example, mRNA transfection, which was found in the working examples to be particularly efficient for expression of exogenous nucleic acids in human cancer cells.

In some embodiments, the provided nucleic acids are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro/ex vivo replication or in vivo replication.

In some embodiments, a nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

### Compositions and Uses

Provided herein are compositions comprising CD4+ CTLs produced by methods of the present disclosure, as well as uses thereof.

In some embodiments, compositions comprising CD4+ CTLs as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses or compositions comprising an effective amount of CD4+ CTLs are also contemplated.

An amount of CD4+ CTLs in a composition or unit dose is at least one cell or is more typically greater than 10² cells, for example, up to 10⁶, up to 10⁷, up to 10⁸ cells, up to 10⁹ cells, or more than 10¹⁰ cells. In some embodiments, the CD4+ CTLs are administered in a range from about 10⁶ to about 10¹⁰ cells/m², such as in a range of about 105 to about 10⁹ cells/m². The number of CD4+ CTLs will depend upon the ultimate use for which the composition or unit dose is intended as well the type of cells included therein. For example, in some embodiments, CD4+ CTLs expressing a TCR specific for a particular antigen will comprise a cell population containing at least 30%, 35%, 40%, 45%, 50%, 55%, 60%, 65%, 70%, 75%, 80%, 85%, 90%, 95% or more of such CD4+ CTLs. For uses provided herein, CD4+ CTLs are generally in a volume of a liter or less, 500 ml or less, 250 ml or less, or 100 ml or less. In embodiments, the density of the desired CD4+ CTLs is typically greater than 10⁴ cells/ml and generally is greater than 10⁷ cells/ml, generally 10⁸ cells/ml or greater. The CD4+ CTLs may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ CD4+ CTLs.

An effective amount of a pharmaceutical composition refers to an amount sufficient, at dosages and for periods of time needed, to achieve the desired clinical results or beneficial treatment, as described herein. An effective amount may be delivered in one or more administrations.

Provided herein are methods for preventing and/or treating a non-malignant disorder, a hyperproliferative disorder, or a relapse of a hyperproliferative disorder, and/or for inducing an immune response against a cell of interest, such as a hyperproliferative cell, comprising contacting undesirable cells expressing an antigen or antigens with CD4+ CTLs described herein targeting such an antigen or antigens (e.g., adoptive cell therapy). In some embodiments, the method comprises administering to a subject a therapeutically effective amount of a composition comprising CD4+ CTLs, such as through autologous, allogeneic, or syngeneic transfer. In some embodiments, CD4+ CTLs described herein may be used to determine the responsiveness to CD4+ CTL therapy for such disorders.

Methods for preparation (e.g., isolation, purification, irradiation, and the like) and administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PloS ONE 8: e61338).

In addition, CD4+ CTL compositions described herein may also be administered in combination therapy with one or more additional agents or therapies to further modulate a desired activity. Additional agents and therapies include, without limitations, Treg modulatory and/or depleting therapy, cancer immunotherapy, immune checkpoint inhibition (e.g., blockade) therapy, chemotherapeutic agents, hormones, antiangiogens, radiolabeled compounds, surgery, cryotherapy, and/or radiotherapy. The preceding treatment methods may be administered in conjunction with other forms of conventional therapy (e.g., standard-of-care treatments for cancer well-known to the skilled artisan), The CD4+ CTL compositions described herein may be administered before, after or consecutively with the additional agent and/or therapy. For example, the CD4+ CTL compositions may be administered with a therapeutically effective dose of chemotherapeutic agent. In another embodiment, the CD4+ CTL compositions are administered in conjunction with an additional therapy, such as immune checkpoint inhibitor therapy, to enhance the activity and efficacy of the additional therapy. The Physicians' Desk Reference (PDR) discloses dosages of additional agents or therapies that have been used in the treatment of various cancers. The dosing regimen and dosages of therapeutic interventions will depend on the particular disorder being treated, the extent of the disorder and other factors familiar to the physician of skill in the art and may be determined by the physician.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro, ex vivo, and in vivo animal studies) and clinical studies and analyzing data obtained therefrom by appropriate statistical, biological, and clinical methods and techniques, all of which can readily be practiced by an ordinarily skilled artisan.

In some embodiments, a subject receiving a unit dose of CD4+ CTLs is receiving or had previously received a transplant, such as a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, CD4+ CTLs may be administered with or shortly after cell transplantation therapy.

In addition, kits and devices comprising CD4+ CTLs described herein are further provided. For example, the kit or device may comprise CD4+ CTLs composition alone or with additional reagents, such as adjuvants, detection reagents, and combinations thereof, packaged in a suitable container and may further comprise instructions for using such reagents. The kit or device may also contain other components, such as administration tools packaged in a separate container. The kit or device may be promoted, distributed, or sold as a unit for performing methods described herein.

### EXAMPLES

### Example 1: CD70-Based Approach to Producing CD4+ CTLs Using B Lymphoma Cells

The present example describes an exemplary CD70-based approach for producing CD4+ CTLs using exemplary murine B-lymphoma cells (A20 cells). Specifically, A20 cells that ectopically express CD70 (amino acid sequence of NP_035747.1 available from GenBank) were found to be sufficient to produce CD4+ CTLs when co-cultured with naïve CD4 T cells in vitro.

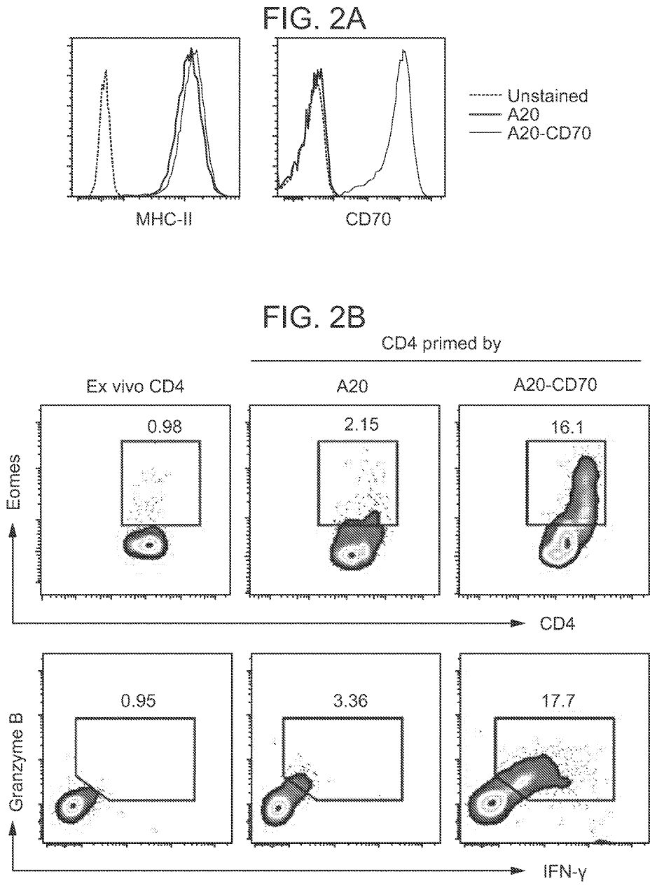

Murine B-celllymphoma model A20 (on a BALB/c background) were used, as these cells express MHC-II, and have no basal expression of CD70 or OX40L. A20 cells were engineered to ectopically express CD70 by transducing with VSVG-pseudotyped retroviruses carrying GFP or CD70-GFP, and GFP+ cells were sorted by FACS at 48 h (hours) after viral transduction. FIG. 2A depicts a FACS analysis of MHC-II and CD70 in A20 cells carrying GFP or CD70-GFP.

Naïve mouse splenic CD4+ T cells were purified using a naïve CD4 T cell isolation kit using magnetic-activated cell sorting (MACS; Miltenyi Biotec) according to manufacturer's protocol. Mouse naïve CD4+ T cells were co-cultured with CD70-GFP-expressing or GFP-expressing A20 cells at a ratio of 2:1 for 6 days or 9 days to generate CD4+ cytotoxic T cells (CD4 CTL).

The present disclosure encompasses a recognition that CD4+ CTLs exert MHC-II-restricted perforin/granzyme B-mediated cytotoxicity; their functional differentiation (including perforin and granzyme B expression) is dependent on the transcription factor Eomes. Representative exemplary CD4+ CTLs were produced by co-culture of naïve CD4+ T cells with CD70-GFP-expressing A20 cells. Briefly, the T cells were stained for CD4, transcriptional factor Eomes, cytotoxic molecules (granzyme B and perforin), and effector cytokines (IFNγ and TNFα). Results of FACS analysis of Eomes, granzyme B and IFN-γ in CD4 cells primed by irradiated CD70-transfected A20 cells when cultured for 6 days and 9 days are shown in FIG. 2B and FIG. 2C, respectively. These results demonstrate that ectopic expression of CD70 in A20 cells makes these cells sufficient to induce formation of CD4+ CTLs in vitro, as indicated by the expression of Eomes, granzyme B (and IFN-γ) in a significant fraction of produced CD4 cells.

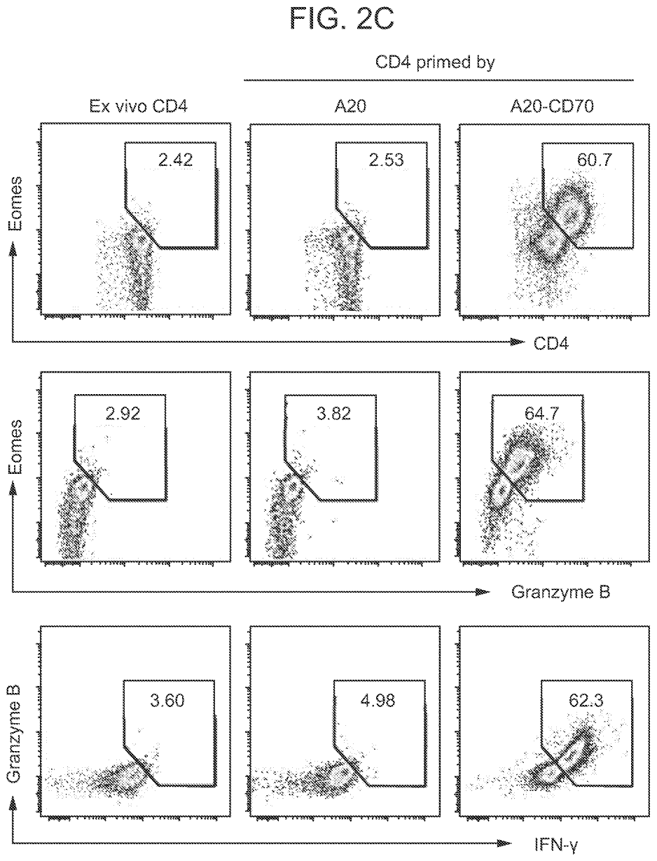

Production of CD4+ CTLs was also assessed using a killing assay. Target cells were labelled with CellTrace™ Violet (Invitrogen) prior to use. CD4+ T cells were co-cultured with 2×10³ target cells at varying effector/target ratios in 96-well round-bottomed plates. The plate was then spun down at 8 g for 2 min followed by incubation for 4-6 h at 37° C. The cultures were stained for active caspase-3 (BD Biosciences); active caspase-3⁺ CellTrace™⁺ cells are defined as apoptotic target cells. Results are shown in FIG. 3. CD4+ T cells primed by A20 cells that ectopically express CD70, but not those that lack CD70, were able to induce specific killing.

These results show that exemplary B-lymphoma cells that ectopically express CD70 are sufficient to enable differentiation of naïve CD4+ T cells to CD4+ CTLs.

### Example 2: CD70 and MHC-II Approach to Producing CD4+ CTLs Using Melanoma Cells

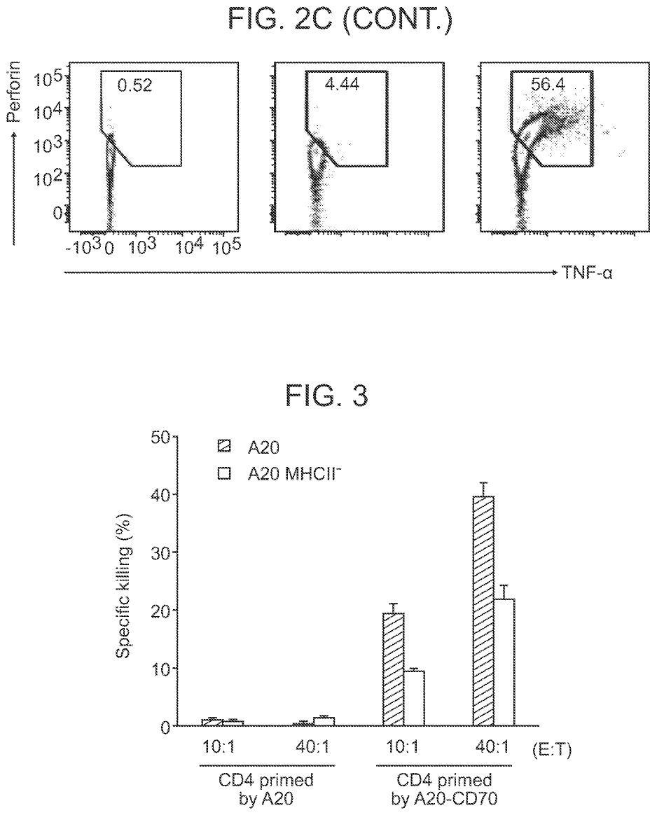

The present example describes an exemplary CD70-based approach for producing CD4+ CTLs using exemplary melanoma cells that have been induced to express MHC-II, as depicted in FIG. 4. Specifically, CD70 was ectopically expressed in interferon gamma (IFNγ) treated murine melanoma cells (B16-F10 cells). B16 cells do not natively express CD70. Exemplary CD70-expressing MHC-II⁺ melanoma cells were found to be sufficient to elicit CD4+ CTLs in vitro.

CD70 cDNA (encoding the amino acid sequence of NP_035747.1 available from GenBank) was cloned into the MSCV-GFP retroviral vector to generate MSCV-CD70-GFP. The MSCV-CD70-GFP or MSCV-GFP retroviral vector was co-transfected with VSVG packaging plasmids (pCMV-VSVG and pKat) into HEK 293T cells. After 48 h, viral supernatants were collected, passed through a 0.45-μm filter, and then used to infect cells immediately or stored at −80° C. for later use. For transduction of B16-F10 cells, cells were infected with VSVG-pseudotyped retroviruses carrying GFP or CD70-GFP (in the presence of 10 μg ml-1 polybrene (Sigma)) for 48 hours, GFP+ cells were sorted by FACS.

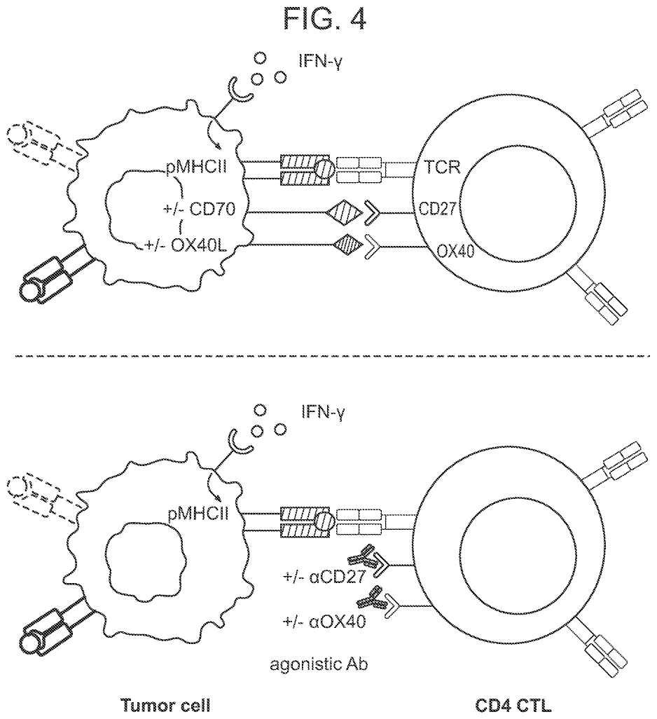

B16 cells are well known to upregulate MHC-II upon exposure to IFN-γ. In the present example, treatment with IFN-γ for 3 days in cell culture induced MHC-II expression in all B16 cells. Mouse IFN-γ (BioLegend, catalog #575306). FIG. 5A shows FACS analysis of MHCII and CD70 expression in GFP or CD70-GFP transduced B16 cells treated with or without IFN-γ (100 ng/mL) for 3 days.

T cell stimulation of exemplary CD4+ T cells that express a transgenic TCR specific to a class II (I-Ab)-restricted epitope from tyrosinase-related protein 1 (Trp1), a TAA expressed in B16 cells (MHC-II⁺, CD70+B16-F10 cells) was assessed.

Trp1 mouse CD4+ T cells were enriched from splenocytes by negative selection with an antibody cocktail (anti-CD11b, anti-CD19, anti-B220, anti-GR-1 and anti-TER-119) using MACS (StemCell Technologies), and then FACS-sorted for CD4+CD25-CD69-. Subsequently, purified I Trp1 CD4 cells were primed in vitro as follows. For CD69 activation assay, naïve Trp1 CD4 cells were cultured in 48-well flat-bottomed plates with different B16 stimulator cells for 18 hours. Results are shown in FIG. 5B. Then CD69 expression was analyzed by FACS. For CD4 CTL generation, naïve Trp1 CD4 cells were cultured in 24-well flat-bottomed plates with irradiated (30 Gy) IFN-γ-pretreated CD70-transfected B16-F10 stimulator cells for 6 days. Then Eomes expression was analyzed by FACS; naïve CD4 T cells from wild-type mice as control. Results are shown in FIG. 5C.

These results show that naïve Trp1-CD4 cells recognized B16 cells in an MHC-II-dependent manner, and that ectopic expression of CD70 in (IFN-γ treated “MHC-II enriched”) MHC-II⁺ B16 cells leads to stronger activation of naïve Trp1-CD4 cells (FIG. 5B) and priming them induces expression of Eomes, the master regulator of CD4 CTL differentiation (FIG. 5C).

These results support that activation of CD4+ T cells using MHC-II⁺ B16 cells expressing CD70 can lead to efficient production of CD4+ CTLs.

### Example 3: Generating Multi-Specific CD4+ CTLs Through Co-Stimulation with Ectopically Expressed Ligand

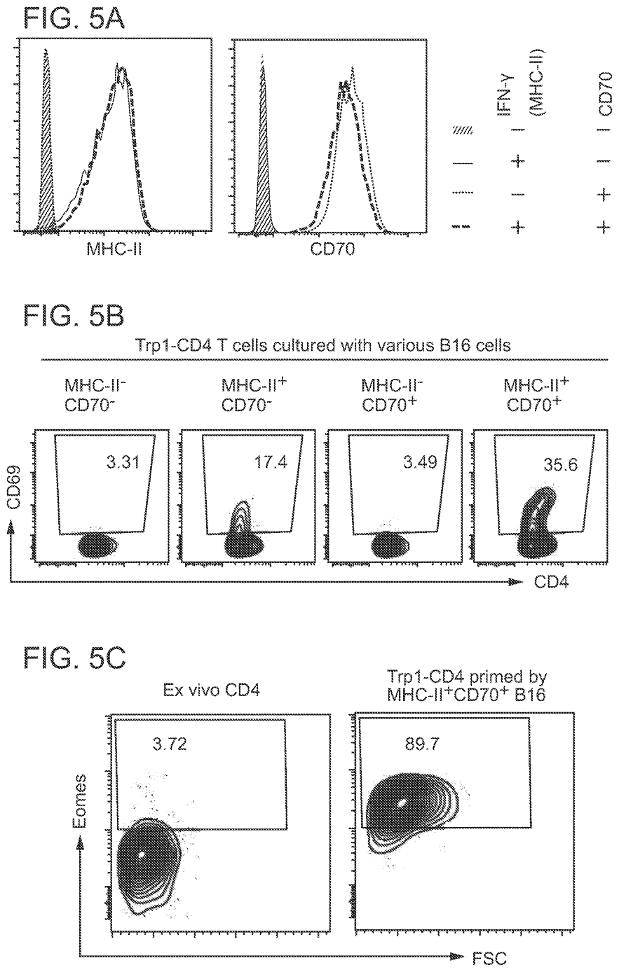

The present example confirms that co-stimulation through ectopic expression of CD70 or OX40L ligands can be used to produce CD4+ CTLs, as depicted in FIG. 4. CD70 or OX40L were overexpressed in exemplary melanoma cells that have also been induced to express MHC-II (interferon gamma (IFNγ) treated B16 cells). Specifically, naïve polyclonal CD4 cells were co-cultured with irradiated MHC-II⁺CD70+ or MHC-II⁺OX40L+B16 cells for 10 days, with mouse IL-2 (final concentration 20 ng/ml) added from 72 hours onward (to facilitate T cell expansion), and then analyzed for the indicated molecules (unprimed CD4+ cells as negative control). As shown in FIG. 6A-FIG. 6E, example OX40L-expressing MHC-II⁺ melanoma cells and example CD70-expressing MHC-II⁺ melanoma cells were both found to be sufficient to elicit CD4+ CTLs in vitro.

These results support that activation of CD4+ T cells using MHC-II⁺ B16 cells expressing either OX40 L or CD70 can lead to efficient production of CD4+ CTLs.

### Example 4: Generating Multi-Specific CD4+ CTLs Through Co-Stimulation with an Agonistic Antibody

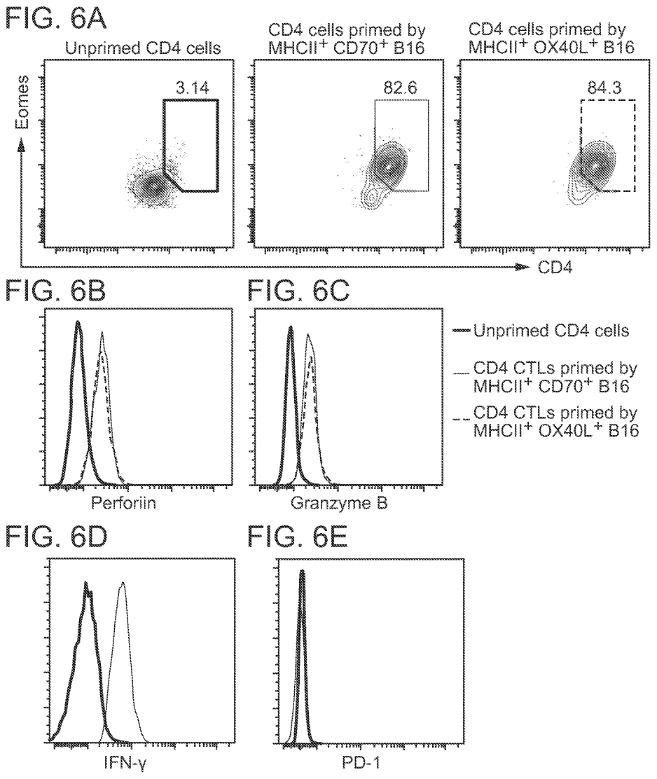

The present example confirms that co-stimulation through agonistic antibodies directed to CD27 or OX40 can be used to produce CD4+ CTLs, as depicted in FIG. 4. Example melanoma cells that have been induced to express MHC-II (interferon gamma (IFNγ)-treated B16 cells) were supplemented with agonistic antibodies to CD27 or OX40. Example anti-mouse CD27 and anti-mouse OX40 (CD134) were from BioXCell, catalog #BE0348, clone RM27-3E5 and catalog #BE0031, clone OX-86, respectively. Specifically, naïve polyclonal CD4 cells were co-cultured with MHC-II⁺ B16 cells and with exemplary αCD27 or αOX40 agonistic antibodies (both at final concentration of 10 μg/ml) for 10 days, with mouse IL-2 (final concentration 20 ng/ml) added from 72 hours onward, and then analyzed for the indicated molecules. Unprimed CD4 cells serve as a negative control. Results are shown in FIG. 7. Example MHC-II+ melanoma cells supplemented with anti-CD27 or anti-OX40 agonistic antibodies were both found to be sufficient to elicit CD4+ CTLs in vitro.

The present disclosure encompasses a recognition that a benefit of using agonistic antibodies for co-stimulation is that the timing of stimulation can be adjusted. The effect of altering the timing of stimulation using anti-CD27 or anti-OX40 agonistic antibodies was also assessed.

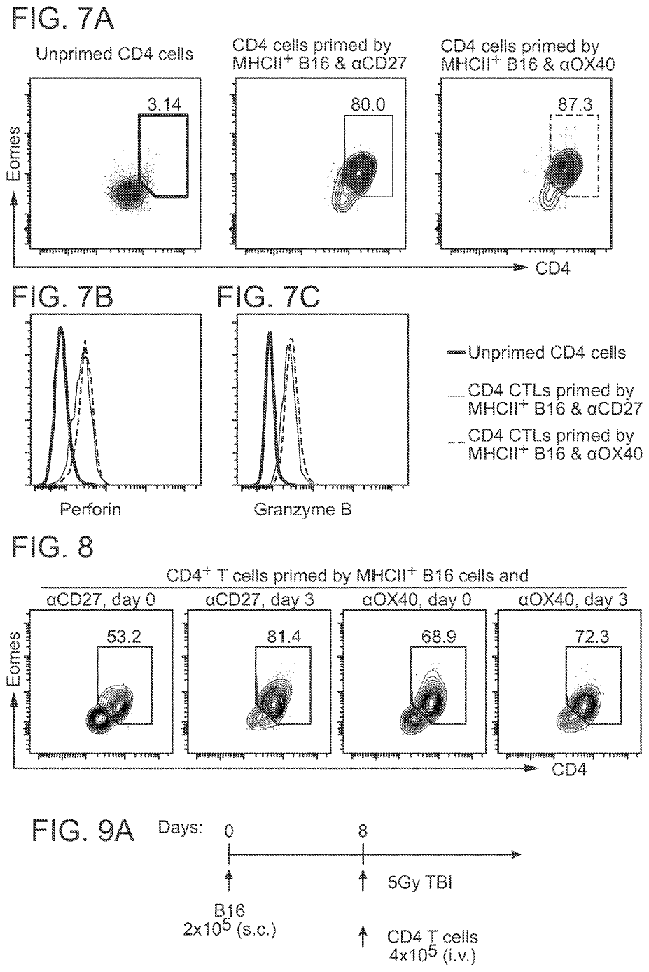

Specifically, naïve polyclonal CD4 cells were co-cultured with MHC-II⁺ B16 cells with the αCD27 or αOX40 agonistic antibody described above added from day 0 or day 3, mouse IL-2 added from day 3 onward, and analyzed on day 10 for the indicated molecules. MHCII+ B16 cells are B16-F10 (from ATCC) treated with mouse IFNg (100 ng/ml, 4 days). Mouse IL-2 (final concentration 20 ng/ml) were added 72 h after co-culturing; αCD27 or αOX40 agonistic antibody was added (final concentration 10 μg/ml) at the indicated timepoint. Surprisingly, increased activation was observed in co-cultures with MHC-II⁺ B16 cells and αCD27 or αOX40 agonistic antibody beginning on day 3, relative to those exposed to agonistic antibody beginning on day 0, as shown in FIG. 8.

These results support that activation of CD4+ T cells using MHC-II⁺ B16 cells and an agonistic αCD27 or αOX40 antibody can lead to efficient production of CD4+ CTLs.

### Example 5: Adoptive Cell Therapy (ACT) with Multi-Specific CD4 CTLs Produced by Provided Methods

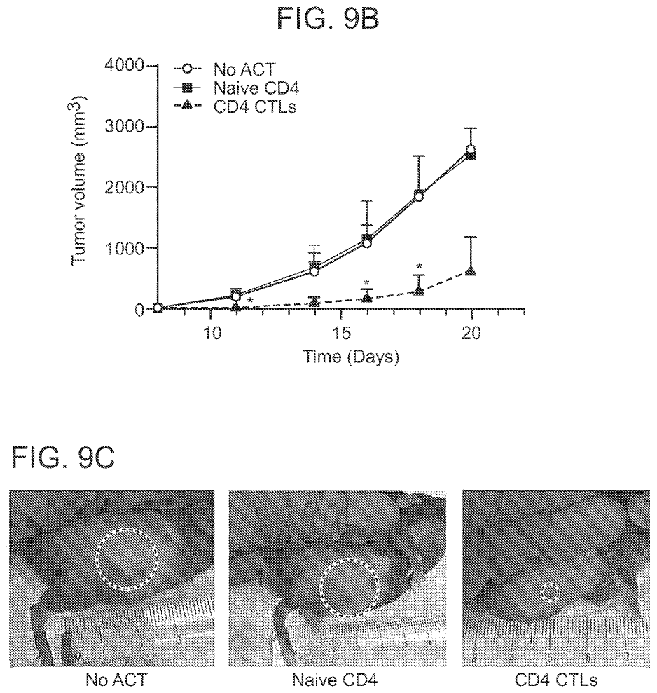

The present example describes an exemplary use of CD4+ CTLs produced using methods described herein in adoptive cell therapy. Specifically, exemplary multi-specific CD4 CTLs primed by MHC-II+CD70+B16 cells were used in adoptive cell therapy in an in vivo mouse model. Mice were treated by the ACT protocol provided in FIG. 9A. Mice were subcutaneously administered B16 cells (2×10\hat{ }5 cells) on day 0. On day 8, mice were irradiated (5Gy total body irradiation (TBI)) and administered by IV CD4 T cells (4×10\hat{ }5 cells). FIG. 9B shows mean tumor volumes over time, in the indicated groups of mice, n=3 mice per group. There was a significant reduction in tumor volume over time relative to the no ACT or naïve-CD4 treated mice. Statistics by one-way ANOVA, *, p<0.05. This effect is also shown in FIG. 9C, which provides representative images on day 18 of tumors treated with the indicated CD4 cells.

These results show that adoptive cell therapy (ACT) with multi-specific CD4 CTLs primed by MHC-II+CD70+B16 cells markedly limits the growth of pre-established B16 tumors.

### Example 6: Upregulation of HLA-II and Ectopic Expression of CD70 in Patient-Derived Melanoma Cells

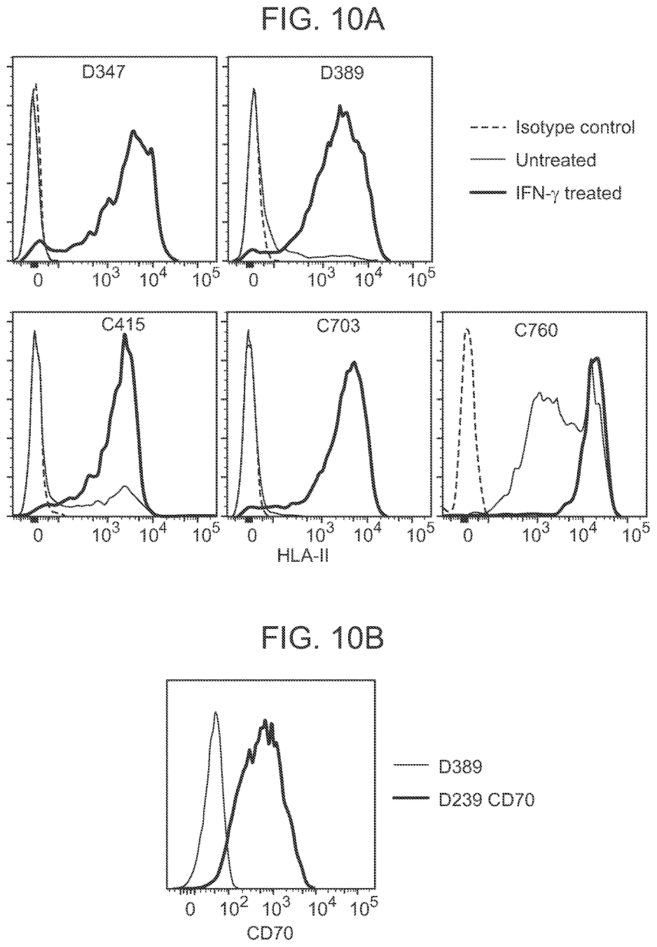

The present example confirms that example patient-derived cancer cells (melanoma cells) can be induced to upregulate HLA-II and ectopically express CD70. IFN-γ treatment with 20 ng/ml hIFN-γ for 3 days upregulates HLA-II expression in 5 different patient-derived melanoma cells, as shown by FACS analysis of HLA-II expression depicted in FIG. 10A. Human IFN-γ (MedChemExpress, catalog #HY-P7025) Patient-derived melanoma cells were also transduced with retroviral vector. cDNA encoding CD70 was cloned into the MSCV-IRES-GFP retroviral vector. The respective retroviral vector was co-transfected with VSVG packaging plasmids into HEK 293T cells. After 48 h, viral supernatants were collected, passed through a 0.45-μm filter, and then used to infect cells immediately or stored at −80° C. for later use. FACS analysis of patient-derived melanoma cells confirmed CD70 expression in CD70 transduced cells, FIG. 10B.

These results show that patient-derived melanoma cells upregulate HLA-II upon IFN-γ exposure and can be induced to express CD70 by gene transduction. These results support the use of primary cancer cells for use in methods for producing CD4+ CTLs described herein.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of producing cytotoxic lymphocytes (CD4+ CTLs) comprising contacting CD4+ T cells with major histocompatibility complex II (MHC II)-expressing cancer cells and (i) one or more agents that promote CD27-mediated co-stimulation of the CD4+ T cells and/or (ii) one or more agents that promote OX40-mediated co-stimulation of the CD4+ T cells, under conditions and for a time suitable for the formation of at least one immune complex between the CD4+ T cells and the cancer cells and promotion of the CD27-mediated co-stimulation of the CD4+ T cells and/or OX40-mediated co-stimulation of the CD4+ T cells, thereby producing the CD4+ CTLs.

2. The method of claim 1, wherein the method does not comprise contacting CD4+ T cells with an OX40-mediated co-stimulation agent.

3. The method of claim 1, wherein the method does not comprise contacting CD4+ T cells with a CD27-mediated co-stimulation agent.

4. The method of claim 1, wherein the method comprises contacting CD4+ T cells with one or more agents that promote CD27-mediated co-stimulation of the CD4+ T cells and one or more agents that promote OX40-mediated co-stimulation of the CD4+ T cells.

5. (canceled)

6. The method of claim 1, wherein the CD4+ T cells are allogeneic, syngeneic, or autologous to the cancer cells.

7. (canceled)

8. The method of claim 1, wherein the cancer cells are or have been induced to express MHC II through contact with interferon gamma (IFNγ).

9. The method of claim 1, wherein the cancer cells endogenously express MHCII.

10. The method of claim 1, wherein the cancer cells do not express CD70, optionally wherein the cancer cells do not natively express CD70.

11. The method of claim 1, wherein the cancer cells are derived from a cancer selected from the group consisting of a B cell leukemia, a B cell lymphoma, diffuse large B cell lymphoma (DLBCL), classical Hodgkin lymphoma (cHL), acute myeloid leukemia (AML), a solid tumor, melanoma, bladder cancer, breast cancer, head and neck cancer, hepatocellular carcinoma (HCC), colon cancer, pancreatic cancer, kidney cancer, ovarian cancer, and brain cancer.

12. The method of claim 1, wherein the cancer cells present at least one MHC II-antigen complex on their cell surface, wherein the antigen is a tumor-associated antigen and/or a neoantigen.

13. The method of claim 1, wherein the one or more agents of i) and the one or more agents of ii) comprise
a) the MHC II-expressing cancer cells, wherein the MHC II-expressing cancer cells are or have been engineered to express CD70 on their cell surface;
b) a soluble CD70, optionally wherein the soluble CD70 comprises a CD70 fusion protein (e.g., a CD70-Fc fusion protein);
c) an anti-CD27 agonistic antibody;
d) the MHC II-expressing cancer cells, wherein the MHC II-expressing cancer cells are or have been engineered to express OX40 ligand (OX40L) on their cell surface;
e) soluble OX40L, optionally wherein the soluble OX40L comprises an OX40L fusion protein (e.g., an OX40L-Fc fusion protein); and/or
f) an anti-OX40 agonistic antibody.

14. The method of claim 1, wherein the CD4+ CTLs:
a) express a T cell receptor (TCR) that specifically binds to an MHC II-antigen complex expressed by cancer cells on the cancer cell surface;
b) express a granzyme, optionally wherein the granzyme is granzyme B;
c) express CD69;
d) express a perforin;
e) are Eomes-programmed;
f) secrete cytokine, optionally wherein the cytokine is IFNγ;
g) induce an antigen-specific T cell immune response;
h) are capable of killing cancer cells;
i) directly kill cancer cells that express on their cell surface the MHC II-antigen complex specifically recognized by the CD4+ CTLs;
j) proliferate;
k) persist in vivo; and/or
l) execute CD4+ helper function.

15.-17. (canceled)

18. The method of claim 1, further comprising a step of treating the APCs (e.g., cancer cells) to prevent further proliferation before contact with the CD4+ T cells, optionally wherein the treatment is irradiation.

19. The method of claim 1, further comprising a step of purifying the CD4+ CTLs.

20. The method of claim 1, wherein the CD4+ T cell is contacted ex vivo or in vitro.

21. A composition comprising CD4+ CTLs produced according to a method claim 1.

22. An immunogenic composition or vaccine comprising CD4+ CTLs produced according to a method of claim 1.

23. The immunogenic composition or vaccine of claim 22, wherein the immunogenic composition or vaccine is capable of eliciting an immune response against antigen-expressing cancer cells in a subject, optionally wherein the immune response is i) a T cell response, a CD4+ helper T cell response, a CD4+ cytotoxic T cell response, and/or a CD8+ T cell response and/or ii) selected from the group consisting of T cell expansion, cytokine release, and/or cytotoxic killing.

24. A method of killing cancer cells comprising contacting the cancer cells with the composition of claim 21.

25. A method of treating cancer comprising administering to a subject with the composition of claim 21.

26.-31. (canceled)
